(19) United States
(12) Patent Application Publication
(10) Pub. No.: US 2021/0213203 A1
(43) Pub. Date: Jul. 15, 2021

(54) Active Agent Delivery Devices and Methods for Using the Same

(71) Applicant: iRenix Medical, Inc., Palo Alto, CA (US)

(72) Inventors: Stephen J. Smith, Palo Alto, CA (US); Thomas W. Chalberg, JR., Palo Alto, CA (US)

(21) Appl. No.: 17/254,514

(22) PCT Filed: Jun. 27, 2019
(86) PCT No.: PCT/US2019/039443

§ 371 Date: Dec. 21, 2020

Related U.S. Application Data
(60) Provisional application No. 62/694,794, filed on Jul. 6, 2018.
(60) Provisional application No. 62/724,535, filed on Aug. 29, 2018.

(51) Int. Cl. A61M 5/315; A61M 5/32; A61M 5/00; A61M 5/31

(52) U.S. Cl. CPC A61M 5/315; A61M 5/3202; A61M 2005/3126; A61M 2210/0612; A61M 5/002

(57) ABSTRACT

Therapeutic agent delivery devices are provided. Aspects of the devices include a syringe that is not surface sterilized, a tip that includes a sterile tissue contacting surface and a needle operably coupled to the syringe and the tip. Also provided are methods of using the devices.

### CROSS REFERENCE TO RELATED APPLICATIONS

Pursuant to 35 U.S.C. § 119 (e), this application claims priority to the filing date of the U.S. Provisional Patent Application Ser. No. 62/694,794, filed Jul. 6, 2018 and U.S. Provisional Patent Application Ser. No. 62/724,535, filed Aug. 29, 2018; the disclosure of which applications are herein incorporated by reference.

### GOVERNMENT RIGHTS

This invention was made with Government support under contract R44 EY028495 awarded by the National Institutes of Health. The Government has certain rights in the invention.

### INTRODUCTION

Pain is a major limiting factor in many common procedures performed in the inpatient and ambulatory care settings. A very abbreviated list of such procedures includes skin biopsy, fine needle aspiration biopsy, IV insertion, vaccination, injections (including injection of anesthetics and gasses), blood draws, central line placements, and finger and heal pricks for blood analysis (glucose measurement). Pharmacologic anesthesia is a primary method of pain reduction, but the delivery of local pharmacologic anesthesia usually requires a painful injection.

The ocular surface is a tissue surface to which therapeutic agents may be delivered. The ability to deliver medication directly into the eye via intravitreal injection therapy (IVT) has transformed the treatment landscape of a number of previously blinding diseases, including macular degeneration and diabetic retinopathy. The success of these therapies in preventing blindness has resulted in a dramatic increase in the number of intravitreal injections performed, with an estimated 4.1 million injections given in the United States alone in 2013. The number of indications for IVT continues to expand, increasing utilization of this therapy significantly every year. The primary limitations of IVT are patient discomfort, ocular surface bleeding, corneal toxicity, and the time constraints of treating the vast number of patients requiring this therapy. These drawbacks relate to the difficulty of delivering ocular anesthesia to the highly vascularized ocular surface.

To give an ocular injection, the physician first provides ocular surface anesthesia by one or more of a number of methods, including the following: topical application of anesthetic drops; a subconjunctival injection of lidocaine; placement of cotton tipped applicators (commonly called a “pledget”) soaked in lidocaine over the planned injection site, application of topical anesthetic gel, or some combination of these. Following ocular anesthesia, the physician or an assistant sterilizes the periocular region by coating it in betadine or a similar antiseptic. Optionally, an eyelid speculum is placed, and the physician marks the location of the injection using calipers that guide placement of the needle. The ocular surface is again sterilized, and the physician gives the injection. Current methods of local anesthesia have unique drawbacks and patients often experience discomfort during and after intraocular injections.

### SUMMARY

Active agent delivery devices are provided. Aspects of the devices include a syringe that is not surface sterilized, a tip that includes a sterile tissue contacting surface and a needle operably coupled to the syringe and the tip. Also provided are methods of using the devices.

### BRIEF DESCRIPTION OF THE FIGURES

FIG. 1 provides a view of a hand-held active agent delivery device according to an embodiment of the invention.

FIG. 2 provides a close-up view of the distal end of the device shown in FIG. 1.

FIGS. 3A and 3B provide views of a tip according to an embodiment of the invention.

FIGS. 4A and 4B provide views of a pre-filled syringe according to an embodiment of the invention.

FIG. 5 provides a view of a docking station according to an embodiment of the invention.

FIGS. 6A to 6I illustrate an active agent administration protocol according to an embodiment of the invention.

FIGS. 7A and 7B provide views of kits according to two different embodiments of the invention.

### DEFINITIONS

As used herein, the term “tissue” refers to one or more aggregates of cells in a subject (e.g., a living organism, such as a mammal, such as a human) that have a similar function and structure or to a plurality of different types of such aggregates. Tissue may include, for example, organ tissue, muscle tissue (e.g., cardiac muscle; smooth muscle; and/or skeletal muscle), connective tissue, ocular conjunctival tissue, nervous tissue and/or epithelial tissue.

The term “subject” is used interchangeably in this disclosure with the term “patient”. In certain embodiments, a subject is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult. While the devices and methods described herein may be applied to perform a procedure on a human subject, it is to be understood that the subject devices and methods may also be carried out to perform a procedure on other subjects (that is, in “non-human subjects”).

The term “sterile” is used in conventional sense to denote free from live bacteria or other microorganisms. A “sterile field” is an area within the operating theater/clinic within which only sterile equipment can be used, and into which only those personnel who have gone through surgical scrubbing and the gowning process can enter.

In some instances, the devices or portions thereof may be viewed as having a proximal and distal end. The term “proximal” refers to a direction oriented toward the operator during use or a position (e.g., a spatial position) closer to the operator (e.g., further from a subject or tissue thereof) during use (e.g., at a time when a tissue piercing device enters tissue). Similarly, the term “distal” refers to a direction oriented away from the operator during use or a position (e.g., a spatial position) further from the operator (e.g., closer to a subject or tissue thereof) during use (e.g., at a time when a tissue piercing device enters tissue). Accordingly, the phrase “proximal end” refers to that end of the device that is closest to the operator during use, while the phrase “distal end” refers to that end of the device that is most distant to the operator during use.

Modules are made up of one or more functional blocks which act in concert to perform a particular function, which is the purpose of the module. A given module may be implemented as hardware, software or a combination thereof. In some instances, modules may include a circuitry element, such as an integrated circuit. When present, integrated circuits may include a number of distinct functional blocks, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material.

Furthermore, the definitions and descriptions provided in one or more (e.g., one, two, three, or four, etc.) sections of this disclosure (e.g., the “Descriptions”, “Devices”, “Methods” and/or “Kits” sections below) are equally applicable to the devices, methods and aspects described in the other sections.

### DETAILED DESCRIPTION

Active agent delivery devices are provided. Aspects of the devices include a syringe that is not surface sterilized, a tip that includes a sterile tissue contacting surface and a needle operably coupled to the syringe and the tip. Also provided are methods of using the devices.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Devices

As summarized above, active agent delivery devices are provided. As the devices are active agent delivery devices, they are configured to deliver an amount (e.g., dosage) of an active, e.g., therapeutic, agent to a target tissue, e.g., via a target delivery site, of a subject. As will be reviewed in greater detail below, the active agent may be in any convenient state, such as liquid, solid or semi-solid (e.g., gel), or gas. In some instances, the devices deliver a liquid active agent composition to a target tissue. While the dosage of liquid active agent composition that is delivered to a target tissue by the devices may vary, in some instances the dosage ranges from 5 μl to 100 μl, such as from 10 μl to 50 μl. In yet other instances the dosage may be larger, and in some embodiments ranges from 100 μl to 5000 μl.

As summarized above, aspects of the devices include a syringe that is not surface sterilized and includes a liquid composition of the active agent; a tip that includes a sterile tissue contacting surface; and a needle, operably coupled to the syringe and the tip. Each of these components is now reviewed in greater detail.

The term “syringe” is used in its conventional sense to refer to a device for injecting fluids. In some instances, a syringe includes a body, such as a tubular body, e.g., a calibrated cylindrical barrel (which may be fabricated from glass or plastic), having a plunger at a first or proximal end and an orifice at a second or distal end, e.g., for providing passage of the active agent composition from the inside of the syringe into a needle which may be operably engaged to the distal end of the container. The syringe is configured to hold a desired amount of an active agent composition. While the volume of the syringe may vary, in some instances the volume ranges from 0.1 to 5.0 ml, such as 0.1 to 1.50 ml, including 0.1 to 1.0 ml, e.g., 0.70 to 0.80 ml. In some instances, the volume of the syringe is sufficient to hold an amount of an active agent composition that is greater than the amount which is delivered to a target tissue delivery site during use of the device. While the magnitude of the excess may vary, in some instances the magnitude ranges from 110% to 500% of the delivered volume, such as 120% to 150%. Where the active agent composition is a non-gaseous composition, the amount of any gas, e.g., air, in the reservoir (and other components of the active agent delivery system) may be minimal, where in some instances the amount is 10 μl or less, such as 5 μl or less, 3 μl or less, 2 μl or less, or 1 μl or less. As indicated above, while the dosage of liquid active agent composition that is delivered to a target tissue by the devices may vary, in some instances the dosage ranges from 5 μl to 100 μl, such as from 10 μl to 50 μl. In some instances, the syringe may include a dosing mark, e.g., an optically recognizable identifier that indicates delivery of a dosage of active agent composition. The dosage that is indicated by the dosing mark may vary, and, in some instances, ranges from 10 to 100 μl, such as 50 μl. In yet other instances, the syringe may not include a dosing mark, e.g., where the device is not configured to be manually operated and the motor is configured to actuate the syringe to deliver an accurate, desired dose. The syringe may be fabricated from any convenient material, including glasses (e.g., type 1 class), plastics (such that the container is a polymeric container, e.g., cyclic olefin polymer (COP) and cyclic olefin copolymer (COC)), etc. Suitable materials include, but are not limited to, those described in published PCT application publication nos. WO 2013/178771; WO2015/173260; WO2017/087798 and WO2017/085253; the disclosures of which are herein incorporated by reference.

As summarized above, in some embodiments the syringe is not surface sterilized. As the syringe of these embodiments is not surface sterilized, the outer surface of the syringe is non-sterile. Non-sterile means any that must be considered to be not sterile or cannot be considered to be sterile, e.g., because of the history of the surface, such as the handling/processing history of the surface (for example as determined by standard medical procedures). In some instances, the non-sterile surface is one that has not been treated so that it is free of live bacteria or other microorganisms (for example, the surface has not undergone a sterilization procedure, such as a heat sterilization procedure (e.g., steam exposure) or chemical sterilization procedure, (e.g., exposure to ethylene oxide gas, exposure to hydrogen peroxide gas plasma, peracetic acid immersion, ozone exposure, etc.). In some instances, the non-sterilized surface has at least one live bacteria or other microorganism present thereon. While the outer surface of the syringe is not sterile, the interior of the syringe, as well as the contents thereof, e.g., liquid active agent composition, are sterile. In yet other embodiments, the syringe may be surface sterilized.

Any desired active agent composition may be present in a container (i.e., reservoir) of the syringe or drug container, including a sleeve to hold solid implants, where an active agent composition may include a single active agent or combination of two or more difference active agents, as desired. An active agent is any component that provides pharmacological activity or other direct effect in the diagnosis, cure, mitigation, treatment, or prevention of disease, or affects the structure or any function of the body of man or animals, and may be a liquid, gel, or solid. A type of active agent that may be present in a given active agent composition is a therapeutic agent, which is an agent that may be used in treating, remediating, or curing a disorder or disease. Active agents may vary, where examples of active agents include, but are not limited to, small molecule active agents, polypeptide active agents, e.g., antibodies and binding fragments thereof, fusion proteins, etc., nucleic acid active agents, cellular active agents, etc. Examples of therapeutic active agents that may be present include, but are not limited to: steroids, such as cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotic agents, such as bacitracin, besifloxacin, levofloxacin, moxifloxacin, sulfacetamide, tobramycin, cefazolin, cephradine, cefaclor, cephapirin, ceftizoxime, cefoperazone, cefotetan, cefuroxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof; vascular endothelial growth factor (VEGF) modulators, e.g., VEGF inhibitors or antagonists, such as tyrosine kinase inhibitors, VEGF specific binding agents, e.g., VEGF antibodies or binding fragments thereof, VEGF binding fusion proteins, and the like; platelet derived growth factor (PDGF) modulators, e.g., PDGF inhibitors or antagonists, such as PDGF specific binding agents, e.g., PDGF antibodies or binding fragments thereof, PDGF binding fusion proteins, and the like; angiopoietin (ANG) modulators, such as ANG2 modulators, e.g., ANG2 inhibitors or antagonists, such as ANG2 specific binding agents, e.g., ANG2 antibodies or binding fragments thereof, ANG2 binding fusion proteins, and the like; combined ANG2 and VEGF inhibitors; antibody biopolymer conjugates containing VEGF inhibitors; poly-lactide-co-glycolide acid (PLGA) particles containing sunitinib malate; placental growth factor (PIGF) modulators, e.g., PIGF inhibitors or antagonists, such as PIGF specific binding agents, e.g., PIGF antibodies or binding fragments thereof, PIGF binding fusion proteins, and the like; tissue necrosis factor (TNF) modulators, such as anti-TNF alpha agents such as antibodies to TNF-α, antibody fragments to TNF-α and TNF binding fusion proteins including infliximab, etanercept, adalimumab, certolizumab and golimumab; mTOR inhibitors such as sirolimus, sirolimus analogues, Everolimus, Temsirolimus and mTOR kinase inhibitors; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents or intraocular pressure lowering agents, such as prostaglandin analogs, ROK inhibitors, beta blockers, alpha agonists, and carbonic anhydrase inhibitors; multi-specific modulators, e.g., bispecific modulators, such as bispecific binding agents, e.g., bispecific antibodies or binding fragments thereof, including agents that specifically bind to both VEGF and ANG2; aminosterols such as squalamine; antihistamines such as H1-receptor antagonists and histamine H2-receptor antagonists, e.g., loratadine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimeorazine doxylamine, pheniramine, pyrilamine, chlorcyclizine, thonzylamine, and derivatives thereof; tyrosine kinase inhibitors, including receptor tyrosine kinase inhibitors; nucleic acid-based therapeutics such as gene vectors, e.g., plasmids, RNAi agents, e.g., siRNA, shRNA; complement system modulators, e.g., complement system inhibitors, including inhibitors of the alternative complement pathway, such as Factor D, properdin, Factor B, Factor Ba, and Factor Bb, and inhibitors of the classical complement pathway, such as C3a, C5, C5a, C5b, C6, C7, C8, C9 and C5b-9; chemotherapeutic agents, e.g., driamycin, cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof; glucose modulators, e.g., insulin; immune modulators, etc.

The active agent or agents are present in the active agent composition in a therapeutically effective amount. The amount of a given active in an active agent composition may vary, e.g., depending on the particular active agent, target condition, etc., where in some instances the amount of a given active agent ranges from 0.0005 to 10000 mg/ml, such as 10 to 500 mg/ml and including 25 to 200 mg/ml.

In addition the active agent or agents, a given active agent composition include pharmaceutically acceptable delivery vehicle, e.g., a pharmaceutically acceptable aqueous vehicle. For pharmaceutically acceptable aqueous vehicles, in addition to water, the aqueous delivery vehicle may include a number of different components, including but not limited to: salts, buffers, preservatives, solubility enhancers, viscosity modulators, colorants, etc. Suitable aqueous vehicles include sterile distilled or purified water, isotonic solutions such as isotonic sodium chloride or boric acid solutions, phosphate buffered saline (PBS), propylene glycol and butylene glycol. Other suitable vehicular constituents include phenylmercuric nitrate, sodium sulfate, sodium sulfite, sodium phosphate and monosodium phosphate. Additional examples of other suitable vehicle ingredients include alcohols, fats and oils, polymers, surfactants, fatty acids, viscosity modifiers, emulsifiers and stabilizers, antimicrobial agents, pH adjusting agents. The viscosity of a given active agent composition may vary. In some instances the viscosity ranges from 0.005 to 5000 centipoise, such as 1 to 500 centipoise and including 0.5 to 400 centipoise.

Active agent compositions that may be delivered according to embodiments of the invention are also described in U.S. Provisional Application Ser. No. 62/722,447 filed Aug. 24, 2018 and titled “Viscous Active Agent Delivery Devices And Methods For Using The Same”; U.S. Provisional Application Ser. No. 62/722,454 filed Aug. 24, 2018 and titled “Solid Active Agent Formulation Delivery Devices And Methods For Using The Same” and U.S. Provisional Application Ser. No. 62/722,657 filed Aug. 24, 2018 and titled “Subcutaneous Delivery Devices And Methods For Using The Same”, the disclosures of which applications are herein incorporated by reference.

A given syringe may include in the container or reservoir thereof a single active agent composition or two or more distinct active agent compositions. The two or more active agent compositions may differ from each other in terms of active agent(s), delivery vehicle, viscosity, etc. Where a given syringe includes two or more distinct active agent compositions, the distinct active agent composition may be separated from each other by a barrier, such as a frangible barrier, such that each distinct active agent composition is present in its own chamber of the syringe. In such instances, the syringe may have two or more chambers, e.g., one for each distinct active agent composition container therein. During delivery, the barrier(s) may be disrupted to provide for combination of the active agent compositions. For example, where two chambers are separated from each other by a frangible barrier, movement of the plunger during active agent composition delivery may disrupt the frangible barrier, allowing for mixing of the two or more active agent compositions. Where desired, a barrier disrupting mechanism may be employed, e.g., a barrier piercing mechanism, etc.

A given device may include a single syringe filed with one or more active agent compositions, e.g., as described above, or two or more syringes each filled with one or more active agent compositions, such as a device that includes two syringes each filled with a different active agent composition. Where a device includes two or more syringes each filled with a different active agent composition, the differing active agent compositions may differ from each other in terms of active agent(s), delivery vehicle, viscosity, etc. In these embodiments, the devices may be configured to deliver the active agent compositions contained in the distinct syringes as separate compositions to the target tissue, or they may be configured to combine the active agent compositions of the disparate syringes prior to delivery to a target tissue. In embodiments where the devices are configured to deliver the active agent compositions contained in the distinct syringes as separate compositions to the target tissue, each syringe may be operatively coupled to its own needle during active agent composition delivery. Alternatively, each syringe may be operatively coupled to a single needle having a distinct internal lumen or passageway for the contents of each syringe, such as needle having a central divider that defines two passageways fluidically isolated from each other and operably coupled to a syringe of a dual syringe system. In embodiments where the devices are configured to combine the active agent compositions of the disparate syringes prior to delivery to a target tissue, the syringes may be operably coupled to single needle having a single passageway during delivery, such that the contents of the distinct syringes mix upon passage through the needle. Where desired, a mixing element may be provided in the device, e.g., at the interface of the syringes and the needle.

Devices of the invention also include a tip having a sterile tissue contacting surface. As the tissue contacting surface of the tip is sterile, as least the portion of the surface that contacts tissue during use of the device is free of live bacteria or other microorganisms. In some instances, the dimensions of the tissue contacting tip will be such that needle entry occurs at a predetermined distance from the corneal limbus (where distances may be as provided above) when the tissue contacting tip is placed on the ocular surface, where such configurations may negate any need for manual measurement for safe injection distance prior to procedure (intravitreal, intracameral etc.). As such, the tissue contacting tip may be configured to prevent contact with a tissue structure near a target tissue delivery site, e.g., an ocular lens or ocular retina where the target tissue delivery site is an ocular tissue delivery site. For example, the tissue contacting tip may be configured to extend only a certain limiting distance beyond the target tissue contacting end of the body of the tissue delivery component. While this limiting distance may vary, in some instances the limiting distance ranges from 0.5 to 8 mm, such as 3 to 4 mm. While the tissue contacting surface may have any convenient configuration, in some instances the surface is planar. The shape of the tissue contacting surface may vary, where shapes of certain embodiments include circular, oval, triangular, rectangular, including square, hexagonal, octagonal, etc. The area of the tissue contacting surface may also vary, where in some instances the area ranges from 0.5 to 50 mm².

Devices of the invention also include a needle operably coupled to the syringe and the tip. The needle is configured to convey the active agent composition from the syringe to a target tissue, e.g., via a target tissue delivery site. The needle may have any convenient dimensions, and, in some instances, has a gauge ranging from 20 to 35, such as 23 to 35, such as 27 to 35, e.g., 30 to 33, such as 29, 29½, 30, 31, 32, 33 and 34 gauge. The needle may include a single lumen or two or more separate lumens container within a common core, as desired, e.g., where the needle includes two separate lumens contained within a common core and has a gauge ranging from 23 to 30. The needle may be made of any convenient material, e.g., stainless steel, etc. At the proximal end of the needle may be a connector for operably connecting to the syringe. Any convenient connector may be present, where connector may be present, such as a luer connector, e.g., a luer slip or luer lock connector.

As summarized above, the needle is operably coupled to the syringe and the tip. The proximal end of the needle may be operably engaged with the distal end of the syringe such that liquid composition inside of the syringe passes through the distal opening of the syringe and into the inside (i.e., bore) of the needle. The proximal end of the needle may be operably engaged with the distal end of the syringe using any convenient configuration. Examples of suitable configurations include, but are not limited to, press fit configurations, luer fitting configurations, e.g., luer slip or luer lock, etc.

In addition to being operably coupled to the syringe, the needle is also operably coupled to the tip that includes the sterile tissue contacting surface. In some instances, the tip is a structure having a proximal end attached to the needle, e.g., by a suitable fitting, and a distal end extending beyond the distal end of the needle, e.g., by a distance ranging from 2 to 20 mm, where the distal end includes the sterile tissue contacting surface. The tip may be made up of a single component or two or more components operably connected to each other. For example, a tip may include a first proximal component and a second distal component, where the distal component includes the sterile tissue contacting surface and the proximal component includes a fitting for operably coupling to the needle. The proximal component may further house the needle or a portion thereof, such that the proximal component may be considered to be a needle housing. The proximal and distal components may be configured to move relative to each other during use. The distance that the two components may move relative to each other may vary, where in some instances the distance ranges from 1 to 20 mm, such as 2 to 10 mm. In some instances, a locking mechanism maintains the distal end of the needle relative to the distal end of the tissue contacting tip prior to actuation. The locking mechanism may be releasable upon movement of the tissue contacting tip relative to the needle, such as a rotational movement, e.g., of the tissue contacting tip about the central longitudinal of the needle. For example, a tissue contacting tip may be turned, such as a quarter turn, relative to the associated needle which results in removing the lock and allowing the needle to be moved relative to the distal end of the tissue contacting tip. The distal, tissue contacting end of the tip may include an opening dimensioned to provide for passage of the distal end of the needle through the contacting tip during active agent delivery, thereby providing access of the needle directly to a target tissue. Upon actuation, the distal end of the needle moves relative to the distal end of the tissue contacting tip. The distance that the distal end of the needle extends beyond the distal end of the tissue contacting tip, and sterile surface thereof, during actuation and active agent delivery may vary, and, in some instances, ranges from 0.1 to 5 mm, such as 0.5 to 4.0 mm. While the dimensions of the opening, when present, may vary, in some instances the opening has a diameter sufficient to accommodate passage of a needle having a gauge, e.g., as described above. The distal end of the tissue contacting tip may in some instances be fabricated from a thermally conductive material, e.g., a metal or alloy thereof, such as in those instances where the distal end of the tissue contacting tip operatively engages the distal end of a cooling element of a cooling system of the actuator component, e.g., as described in greater detail below. Other suitable materials may also be employed, such as polymeric materials. A given tip may be configured to be operably coupled to a single needle, or two or more needles. For example, a given tip may be configured to operably couple to two needles, e.g., where a device includes two syringes, such as described above.

In some instances, the tissue contacting tip may include one or more filters. The filters may be configured to remove particles or other unwanted components present in the active agent composition prior to delivery to the target tissue delivery site. Such filters may be configured to inhibit passage of particles above a certain pore size from >0.1 μm to >50 μm, such as >5 μm. The one or more filters may be positioned at any convenient location in the therapeutic agent delivery system, e.g., at the exit from the container into the injector, at some point along the injector, at the distal end of the injector, etc.

Where desired, the distal, tissue contacting surface may include a removable cover, e.g., that is present until the device is used to deliver therapeutic agent to a target delivery site. The cover may be configured as a release liner or analogous structure, such that it may be easily removed just prior to use. The cover may be sterile or sanitized as desired, and fabricated from any convenient material, e.g., plastics, etc. It may also take the form of a sterile peel pack, sterile box, etc.

The device may, where desired, include an antimicrobial element. The antimicrobial element may be any convenient element having antimicrobial properties and be positioned at one or more locations of the therapeutic agent delivery system. For example, the antimicrobial element may be positioned at the distal, tissue contacting end of the body in order to provide for at least aseptic conditions during contact of the device to the target tissue delivery site, in order to sanitize the target tissue delivery site, etc. The antimicrobial element may include an antimicrobial agent, which may be present in a holder, such as a matrix material, reservoir, etc. As with the therapeutic agent, the antimicrobial agent, when present, may be present in a composition that is in a variety of different physical steps, including liquid, solid, semi-solid, and gaseous. Antimicrobial agents of interest include, but are not limited to: povidone-iodide (Betadine), chlorhexidine (Nolvasan), ethanol, chlorine dioxide and derivatives, other alcohols, and the like.

The active agent delivery system may, where desired, include an analgesic/anesthetic agent. When present, the analgesic/anesthetic agent may be present in any convenient manner that provides for delivery of the analgesic/anesthetic agent to the target tissue delivery site during use of the device. For example, the analgesic/anesthetic agent may be positioned at the distal, tissue contacting end of the tip in order to provide for at least aseptic conditions during contact of the device to the target tissue delivery site. The analgesic/anesthetic agent may be present in a holder, such as a matrix material, reservoir, etc. As with the therapeutic agent, the analgesic/anesthetic agent, when present, may be present in a composition that is in a variety of different physical steps, including liquid, solid, semi-solid, and gaseous. Analgesic/anesthetic agents of interest include, but are not limited to: lidocaine, benzocaine, prilocaine, lidocaine, dubicaine, mepivacaine, bupivacaine, and the like; naturally-derived products, such as saxitoxin, neosaxitoxin, tetrodotoxin, menthol, eugenol, and cocaine, and the like; etc.

The active agent delivery devices of the invention may be configured for delivery of an active agent to a variety of target tissues and/or delivery sites therefore. Examples of target tissues include both external and internal sites, where internal delivery sites include those sites located in body cavities. External sites may include keratinized sites, as well as sites characterized by cutaneous membranes, mucous membranes, and tissue of the mucocutaneous zone. In some instances, the target tissue is one that accessed via an ocular tissue delivery site, where ocular tissue delivery sites of interest include a region that begins at the corneal limbus and extends anywhere from 1 mm to 10 mm posterior to the limbus, 2 mm to over 8 mm posterior to the limbus, such as 3 mm to 6 mm from the corneal limbus, e.g., 3 to 4 mm from the corneal limbus, e.g., to allow intraocular injection via pars plana or pars plicata. Ocular tissue delivery sites may include cornea, conjunctiva, episclera, and sclera of the eye. Ocular tissue delivery sites of interest include those that provide for intravitreal injection therapy (IVT), retrobulbar injection therapy, subtenon injection therapy, subretinal injection therapy, suprachorodial injection, subconjunctival injection therapy, intracameral injection therapy, and the like.

In some instances, a given active agent delivery device includes only the syringe and needle operably coupled to the tissue contacting tip, e.g., as described above. For example, the device may be configured for manual actuation, such that a user, such as a health care practitioner, can position the sterile tissue contacting surface of the tip onto a target location, e.g., a location of the surface of the eye, move the needle housing relative to the distal tip so as to move the needle through the eye surface into the eye, and then depress the plunger of the syringe so as to move a dosage of an active agent composition from the syringe via the needle into the eye.

In yet other instances, the device may include an actuator which is configured to move the plunger of the syringe during use. In these instances, the syringe and tissue contacting tip, which collectively may be referred to as the active agent delivery system, may be configured to be operably, and in some instances release-ably, engaged in a receiving space of an actuator component of the device. In these instances, the active agent delivery system may include a component of a locking element for release-ably engaging the active agent delivery system in a receiving space of the actuator component of the device. Any convenient locking mechanism may be employed, such as but not limited to: press fit, moveable latch, and the like. The locking element component of the locking element that is present on the active agent delivery component may vary, as desired, and is selected based on the companion element that is present on the actuator.

As such, embodiments of active agent delivery devices include an active agent delivery system and an actuator component, where the active agent delivery system is present in a receiving space, such as a syringe receiving space, of the actuator component and the actuator component includes an active agent delivery system actuator. In some instances, the active agent delivery system is release-ably engaged in the receiving space of the actuator component. Accordingly, in such instances the active agent delivery system is configured to be readily separable from the receiving space of the actuator component without in any way damaging the functionality of the actuator component, such that another active agent delivery system may be positioned in the receiving space of the actuator component. As such, the devices of the present invention are configured so that the actuator component can be sequentially employed with multiple different active agent delivery systems. Of interest are configurations in which the active agent delivery system can be manually operably positioned in the receiving space of the actuator component unit without the use of any tools. In some instances, the device further includes a locking element for release-ably engaging the active agent delivery system in the receiving space of the actuator component of the device. Any convenient locking mechanism may be employed, such as but not limited to: press fit, moveable latch, and the like.

Where a given device includes both an active agent delivery system and an actuator component, the active agent delivery system may further include one or more identifiers. When present, such identifiers may be present on one or more components of the active agent delivery system, such as the syringe, the tip, etc. In some instances, an identifier present on the active agent delivery system is an identifier that is configured to be read by an identifier reader of the actuator component of the device, e.g., as described below. While such reader compatible identifiers may vary, in some instances the identifier is a barcode, such as a linear barcode or a matrix barcode, such as a QR code. In some instances, the reader compatible identifier is a radio frequency identification (RFID) tag, such as a near field communication (NFC) tag, where the RFID tag may be passive or active. Information included in the identifier may include, but is not limited to, identity of the therapeutic agent (brand name and/or generic name), date of manufacture, date of expiry, source of manufacture, dosage amount, drug concentration, intended route of administration, handling and storage information, delivery volume, indication for use, lot number, etc.

In addition to, or instead of, a reader compatible identifier, the therapeutic agent delivery system may include an identifier that is visual identifier, such that it is configured to be read by a health care practitioner. Visual identifiers are identifiers that may be readily understood by a human upon looking at the identifier, such that computer processing of the identifier is not required. Examples of such identifiers include, but are not limited to, text identifiers, color coding identifiers, commonly understood symbols, identifying trademarks, logos, and the like. Information conveyed by the visual identifier may vary as desired, where examples of information that may be conveyed by the visual identifier include, but are not limited to: information about the active agent delivery system or active agent present therein, such as identity of the therapeutic agent (brand name and/or generic name), date of manufacture, date of expiry, source of manufacture, dosage amount, drug concentration, intended route of administration, handling and storage information, delivery volume, indication for use, lot number, etc.

As described above, the actuator component is configured to operably engage with an active agent delivery system such as described above, to produce an active agent delivery device of the invention. Aspects of actuator components according to embodiments of the invention include a body having a proximal end and a distal end, an active agent delivery system receiving space configured to be operably, and in some instances release-ably, engaged with an active agent delivery system, e.g., as described above. Further aspects of the actuator component include an active agent delivery system actuator configured to actuate an active agent delivery system. The actuator may further include one or more components of a pain mitigation system configured to mitigate pain in target tissue delivery site, and in some instances the actuator may include all of the components of a pain mitigation system.

The actuator is an element or subsystem that is configured to actuate the active agent delivery system so as to deliver an active agent to a target tissue delivery site. The nature of the active agent delivery system actuator may vary, e.g., depending on the nature of the active agent delivery system. For example, the active agent delivery system actuator may be configured to provide for control of one or more of angular position, linear position, velocity and acceleration of the needle. In some instances, the actuator, either alone or in conjunction with a guiding element of the active agent delivery system, is configured to provide for an angle of the needle relative the distal, tissue contacting end of the active agent delivery system, that ranges from 0 to 90, such as 75 to 90°. In some embodiments, the active agent delivery system is present in the device at a pre-determined angle, for example, 90 degrees to the biologic tissue when the cold tip is applied to the biologic tissue, so that when the device tip is placed on the eye abutting the limbus and causing very slight indentation of the ocular surface 360 degrees around the tip, the needle tip will reproducibly be inserted into the eye at a defined, safe angle posterior to the limbus of the eye to avoid the danger of striking the retina, zonules, or lens. In some instances, the actuator provides for a velocity of introduction of the injector into a target tissue delivery site that ranges from 0.1 to 100 mm/sec, such as 1 to 10 mm/sec, and including 3.5 to 9 mm/sec. The active agent delivery system actuator may be configured to provide for control of release of an active agent composition from the syringe. The actuator may be configured to provide for controlled removal of the needle from the target tissue delivery site. In some such instances, the actuator may be configured to withdraw the needle from a target tissue delivery site at a velocity ranging from 0.1 to 100 mm/sec, such as 1 to 10 mm/sec, and including 3.5 to 9 mm/sec. In some instances, the actuator is configured to prime the tissue injector, e.g., where the active agent delivery system includes an amount of gas, e.g., air (such as in the form of bubbles) and the actuator removes the gas from the system, e.g., by causing the gas to evacuate from the system via the needle. In some instances, the actuator is configured to sequentially move the drug container in a first priming motion and a second injection motion. In some instances, the actuator is further configured to withdraw the needle back into the device following injection of the active agent composition

The actuator may vary as desired. Examples of actuators that may be employed in embodiments of the invention and present in the actuator component include, but are not limited to: motorized actuators (including those that include a micro-motor such as a stepper motor, DC motor, brushed motor, or brushless motor), as well as non-motorized actuators, e.g., pneumatic powered actuators, hydraulically power actuators, spring-loaded actuators, manually operated actuators, e.g., plunger comprising actuators, and the like. The functionality of the actuator may be controlled by one or more modules, as desired. The actuator can be configured to modify the speed and depth of drug injection.

In addition to the actuator, the actuator component may include one or more components of, including all of, a pain mitigation system. The actuator may provide for pain mitigation, such that the actuator is configured to alleviate pain associated with delivery of an active agent to the target tissue delivery site by the device. While the magnitude of pain mitigation may vary, in some instances the magnitude of pain mitigation is 5% or more, such as 10% or more, and including 20% or more, as compared to a suitable control (such as identical delivery without pain mitigation). A pain mitigation system is a system that provides for pain alleviation during delivery of an active agent to a target delivery site, as discussed above. The pain mitigation system may vary as desired, where pain mitigation systems finding use in devices of the invention include both anesthesia producing systems (i.e., systems that result in at least some degree of, if not complete loss of, sensation in the target tissue delivery site, e.g., via blockage of all feeling in the target tissue delivery site) and analgesia producing systems (i.e., systems that result in relief of pain without total loss of feeling in the target tissue delivery site).

In some instances, an anesthesia producing system is a cooling system, i.e., a system that decreases the temperature of the target tissue delivery site by an amount sufficient to produce the desired anesthesia in the target tissue delivery site. The cooling system may vary, and, in some instances, is a system that provides for contact of a cold element (e.g., a cold tip or cold tissue engager (such as a tissue contacting tip, e.g., as described in greater detail below) with the target tissue delivery site. The cold element (which may be a component of a tissue engager, e.g., as described in greater detail below) of the cooling system may vary, and, in some instances, is an element that is configured to maintain a temperature of between −80° C. to +5° C., such as −20° C. to 0° C., such as −20° C. to −5° C. and including −15° C. to −5° C. when contacted with the target tissue delivery site. During a given delivery method, a tissue engager may maintain a constant temperature or cycle through one or more distinct temperature ranges, as desired. For example, a tissue engager may be configured to have a temperature that falls within a first range (e.g., as described above) to provide for desired cryoanesthesia during therapeutic agent delivery, and then cycle to a second, warmer temperature prior to remove of the device, such as a temperature ranging from 0 to −5° C., such as 0 to −2.5° C., including 0 to −1° C. In another embodiment, the cooling may be passive, with the temperature warming up based on factors like the amount of time the device is not attached to the cooling base station, the amount of time the device tip is placed against biological tissue, and the temperature of said biological tissue. Where the target tissue delivery site is an ocular tissue delivery site, e.g., as described elsewhere, delivery of cooling to cause rapid vasoconstriction enables a reduction in the occurrence of ocular surface bleeding and prevents repeated vascular trauma with long term circulatory compromise.

Specific cooling systems of interest that may find use in anesthesia producing pain mitigation systems may vary, where cooling systems of interest include, but are not limited to: thermoelectric cooling systems, liquid evaporation cooling systems, solid sublimation cooling systems, Joule-Thompson cooling systems, thermodynamic cycle cooling systems, endothermic reaction cooling systems, low-temperature substance cooling systems, and the like.

In some instances, the pain mitigation anesthesia producing system is a thermoelectric cooling system, e.g., one that includes one or a combination of thermoelectric (Peltier) devices or units. While thermoelectric cooling systems employed in embodiments of devices of the invention may vary, in some instances the thermoelectric cooling systems include a cold tip that is configured to contact a target tissue delivery site (and therefore may also be referred to as a tissue engager), as well as one or more of a power source, a controller, a cooling power concentrator, one or more Peltier unit modules, and a heat sink (which may be a solid material or include one a fluid, such as a liquid, phase in a container. It should be understood that, in some embodiments, a given thermoelectric cooling system may include a heating element (not shown) that operates in conjunction with the cooling elements to precisely maintain a desired temperature and/or heat flux. Further details regarding embodiments of thermoelectric cooling systems that may be employed in devices of the invention are provided in U.S. Pat. No. 9,956,355; the disclosure of which is herein incorporated by reference.

In yet other instances, the cooling system may include a substance having a melting temperature of 0° C. or lower, such as a phase change material, e.g., PIusICE E-11 or E-15 phase change materials (PCM Products Ltd.). As reviewed above, other non-thermoelectric cooling pain mitigation systems may be employed, such as but not limited to: liquid evaporation cooing system, solid sublimation cooling system, Joule-Thompson cooling system, thermodynamic cycle cooling system, an endothermic reaction cooling system and a low-temperature substance cooling system.

Instead of cooling systems, other types of anesthesia producing systems may be employed as pain mitigation systems. Such anesthesia systems include, but are not limited to: system that deliver an anesthetic agent, such as but not limited to: sodium-channel blockers, e.g., as amino amides or amino esters (such as proparacaine, tetracaine, or lidocaine drops, gels, or creams), naturally-derived agents, such as saxitoxin, neosaxitoxin, tetrodotoxin, menthol, eugenol, and cocaine; and the like.

Also of interest as pain mitigation systems are analgesia producing systems, e.g., as summarized above. Examples of analgesia producing systems finding use in embodiments of devices of the invention include application of agents considered above as local anesthetics. They may also include, but are not limited to, additional techniques such as electrical stimulation (Campbell and Taub, Arch Neurol. 1973; 28(5):347-350) and the like.

As indicated above, in some instances, the actuator component may include a component of a locking element for release-ably engaging the active agent delivery system in the receiving space of the actuator component of the device. As reviewed above, any convenient locking mechanism may be employed, such as but not limited to: press fit or snap on, and the like. The locking element component of the locking element that is present on the actuator component may vary, as desired, and is selected based on the companion element that is present on the actuator.

In some instances, the actuator component includes an identifier reader for reading an identifier of an active agent delivery system release-ably engaged with the actuator component. The identifier reader may vary, as desired, depending on the nature of the identifier that is associated with the therapeutic agent delivery component. For example, where the identifier is a barcode, the identifier reader of the actuator may be any convenient barcode or QR code scanner. Likewise, where the identifier is a radiofrequency identifier, the identifier reader of the actuator may be any convenient RFID reader or NFC reader. The identifier reader, when present, is located on the actuator at a position such that it is reading relationship with the identifier of a therapeutic agent delivery component when release-ably engaged with the actuator.

Where the actuator component includes an identifier reader, in some instances the actuator is configured to be active only when the identifier reader detects an acceptable identifier. An acceptable identifier may be an identifier that imparts one or more types of information upon which acceptability may be based, such as but not limited to: whether the active agent delivery system is filled with the correct active agent, whether the active agent delivery component is expired, where the active agent delivery component is manufactured by an acceptable, authentic source; whether the active agent delivery component has been previously registered as lost, etc. In such instances, the reader may be coupled to an actuator control element that only enables one or more actuator components, such as the therapeutic agent delivery system actuator, the pain mitigation system, etc., when an acceptable identifier is read by the identifier reader. As such, where an unacceptable identifier is read by the reader, the reader may send a single to the controller that disables one or more of the actuator components. Alternatively, where an unacceptable identifier is read by the reader, the reader may send a single to the controller that one or more of the actuator components should not be enabled.

Actuator components of the invention may further include a communications module, which module is operably coupled to one or more components of the actuator and provide for data transfer therefrom to another component, e.g., an external device, etc. The communications module may be configured to provide for the transfer of data in a wired or wireless mode, as desired. For example, the communications module may be configured to wirelessly transfer data, e.g., with a networked device, while be used, and then transfer data using a wired configuration when docked at a docking station, such as described below. Communications modules of the actuators may be configured, e.g., via hardware and/or software implementation, to perform desired communications functions, e.g., to receive data from an actuator element, to transfer data, e.g., to a USB port for wired communications or a wireless transmitter for wireless communications, etc. Communications modules (as well as any other modules described herein, such as actuator controller modules, etc.) are made up of one or more functional blocks which act in concert to perform a particular function, which is the purpose of the module. A given communications module may be implemented as hardware, software or a combination thereof. In some instances, the communications module may include a circuitry element, such as an integrated circuit. When present, integrated circuits may include a number of distinct functional blocks, i.e., modules, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material.

Where desired, actuator components may include a variety of different types of power sources that provide operating power to the actuator component in some manner. The nature of the power source may vary, and may or may not include power management circuitry. In some instances, the power source may include a battery. When present, the battery may be a onetime use battery or a rechargeable battery. For rechargeable batteries, the battery may be recharged using any convenient protocol. In some applications, the actuator may have a battery life ranging from 0.1 to 100 hrs, such as 0.5 to 10 hrs or 1 hour to 5 hours.

In certain instances, the actuator of the invention includes an updatable control module, by which is meant that the actuator is configured so that one or more control algorithms of the actuator may be updated. Updating may be achieved using any convenient protocol, such as transmitting updated algorithm data to the control module using a wire connection (e.g., via a USB port on the device) or a wireless communication protocol. The content of the update may vary. In some instances, a actuator component is updated to configure the unit to be used with a particular therapeutic agent delivery component. In this fashion, the same actuator component may be employed with two or more different therapeutic agent delivery components that may differ by from each other in one more ways, e.g., identify of therapeutic agent, manufacturer of therapeutic agent delivery component, etc. The update information may also include general functional updates, such that the actuator component can be updated at any desired time to include one or more additional software features and/or modify one or more existing programs of the device. The update information can be provided from any source, e.g., a particular elongated member, the internet, etc.

The actuator component may include one or more safety mechanisms, e.g., in addition to or instead of, the identifier/reader compatibility mechanism as described above. In some embodiments, the therapeutic agent delivery system actuator will provide for actuation only if a switch is depressed continuously during the injection process. In some embodiments, there will be a safety mechanism to halt injection.

In some instances, the actuator components may include a display. By display is meant a visual display unit, which may include a screen that displays visual data in the form of images, lights, and/or text to a user. The screen may vary, where a screen type of interest is an LCD screen. The display, when present, may be integrated with the actuator component. As such, the display may be an integrated structure with the actuator component, such that it cannot be separated from the actuator component without damaging the monitor in some manner. The display, when present will have dimensions sufficient for use with the actuator, where screen sizes of interest may include 100 cm² or smaller, such as 20 cm² or smaller, etc. The display may be configured to display a variety of different types of information to a user, where such information may include devices settings, including tip temperature, time of cooling application, therapeutic agent identification, and therapeutic agent expiration date, etc. In some instances, the actuator components may include an LED-based light system to inform the user of the status of the device, including cooling parameters, syringe position, injection status, and battery indicators.

The entire active agent delivery device may be configured for single use, such that the entire active agent delivery device is disposable. Alternatively, one or more components of the active agent delivery device may be reusable. For example, the actuator component may be reusable while the active agent delivery system and components thereof, e.g., syringe, needle, tissue contacting tip, etc., may be single use.

The various device components of the invention may be fabricated using any convenient materials or combination thereof, including but not limited to: metallic materials such as tungsten, copper, stainless steel alloys, platinum or its alloys, titanium or its alloys, molybdenum or its alloys, and nickel or its alloys, etc.; polymeric materials, such as polytetrafluoroethylene, polyimide, PEEK, and the like; ceramics, such as alumina (e.g., STEATITE™ alumina, MAECOR™ alumina), etc. The drug reservoir can be made of plastic, such as polypropylene or polystyrene, or any material commonly used for syringes and the like. It can also be made of glass, including type 1 glass, as is commonly used for long-term storage of drugs and biologics. Alternatively, it can be made of non-leachable plastic materials that are used for long-term storage of drugs or biologics, such as cyclic olefin copolymer (Crystal Zenith) and the like.

Active agent delivery devices as described herein may be handheld. In such embodiments, as the devices are handheld, they are configured to be held easily in the hand of an adult human. Accordingly, the devices may have a configuration that is amenable to gripping by the human adult hand. The weight of the devices may vary, and, in some instances, may range from 0.05 to 3 pounds, such as 0.1 pounds to 1 pound. Handheld devices of the invention may have any convenient configuration, where examples of suitable handle configurations are further provided below.

### Specific Embodiments

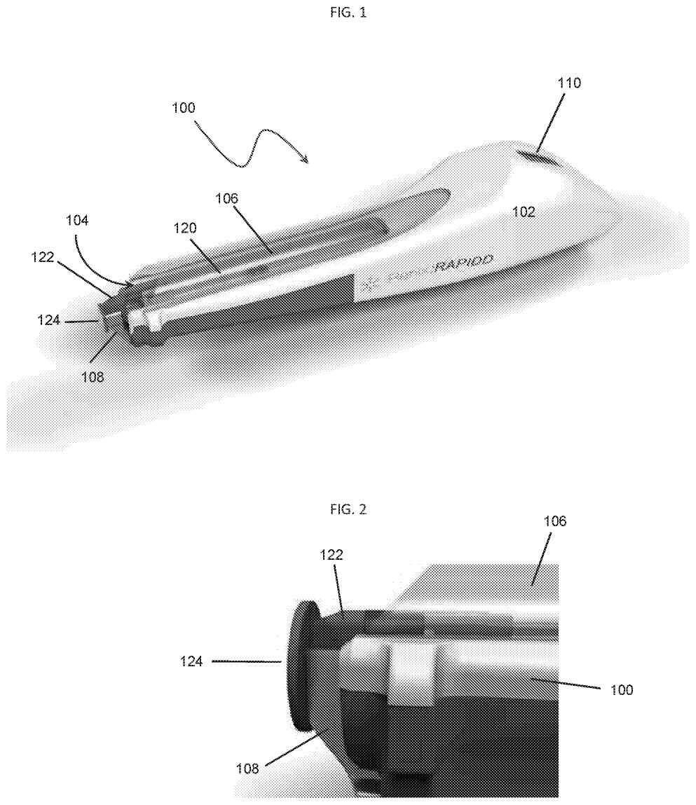

FIG. 1 provides a view of a hand held therapeutic agent delivery device according to an embodiment of the invention. As shown in FIG. 1, device 100 includes an actuator component 102 that includes an active agent delivery system receiving space 104 and a transparent cover for the receiving space 106. Also shown is the cooling tip 108 of a pain mitigation system of the actuator component 102. The actuator component 102 also includes an LED 110. Present in the receiving space 104 is active agent delivery system 120, which includes a tip 122 having a sterile, planar tissue contacting surface 124.

FIG. 2 provides a close-up view of the distal end of the device 100 shown in FIG. 1. As shown in FIG. 2, distal end of actuator 100 includes the tip 122 operably engaged with the cooling arm 108 of the pain mitigation system of the actuator 100. The tissue contacting surface 124 of the tip 122 is sterile, while the cooling arm 108 is not.

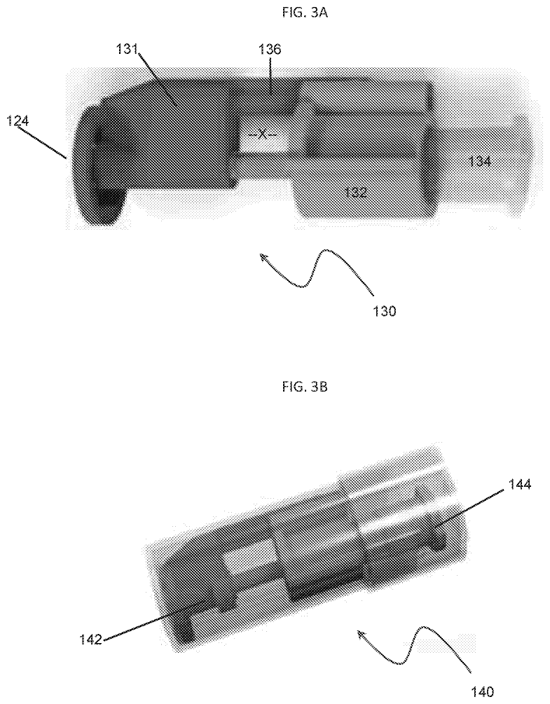

FIG. 3A provides a close-up view of tip component 130, which includes a tip 122 operably coupled to a needle. As shown in FIG. 3A tip component 130 is made up of two distinct components, i.e., a distal component 131 and a proximal component 132. Distal component 131 includes the sterile tissue contacting surface 124, and may be referred to as the tissue contacting component. Proximal component 132 houses the needle and may also be referred to as the needle housing. As shown, needle housing 132 is in sliding relationship with distal component 130, as provided by rail 136 of distal component 132. The distance X may vary, and, in some embodiments, ranges from 1 to 20 mm, such as 2 to 10 mm. Also shown is the luer fitting 134 of the needle present in the needle housing, wherein luer fitting 134 is configured to operably engage with a syringe. During use, needle housing 132 and distal component 131 are moved together such that the distance X decreases to 5 mm or less, such as 2 mm or less, where in some instances X becomes 0 mm, and the distal end of the needle extends through the passageway of the sterile tissue contacting surface 124. Where desired, before use tip component 122 may be provided in a sealed enclosure, such as illustrated in FIG. 3B. As shown in FIG. 3B, tip component 130 is present in a tip component housing 140 made up of a container 142 and a cap 144. Container 142 and cap 144 may fit together using any convenient approach, such as snap fit, screw fit, etc., and may provide for a sterile enclosure for housing the tip.

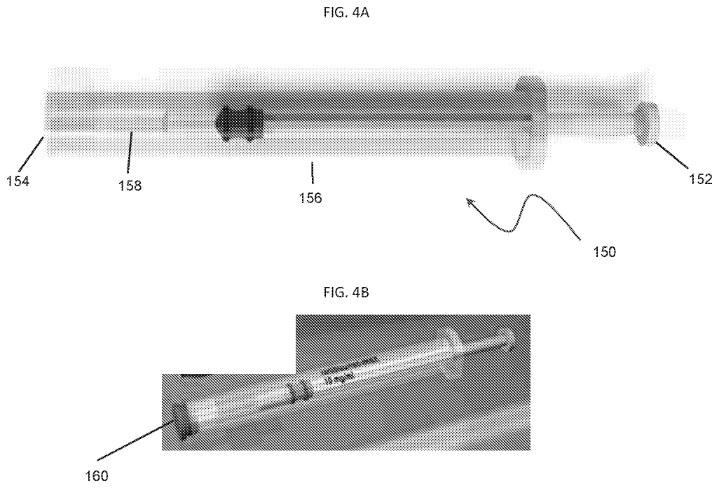

FIG. 4A provides a view of the syringe of device 100. As shown in FIG. 4A, syringe 150 is made from a transparent material, e.g., glass or plastic, and includes plunger 152 at its proximal end, and distal end 154 configured to operably couple to proximal component 132 of the tip 122 shown in FIG. 3A. Outer surface 156 is not sterile, while interior 158 which contains an active agent composition is sterile. Prior to use, the distal end of the syringe 150 may be sealed with a cap 160, e.g., in order to preserve the sterility of the interior of the syringe, e.g., as shown in FIG. 4B.

### Docking Stations and Systems Including the Same

Aspects of the invention include docking stations that are configured to dock an actuator component, and systems that include a docking station and an actuator component. A docking station is a base unit or analogous device that is configured to engage with an actuator component, e.g., as described above. When engaged with an actuator component, the docking station may perform one or more functionalities, which functionalities may include, but are not limited to: maintaining a pain mitigation system in a desirable state (for example maintain a cooling system at a desired temperature); transferring data between the actuator component and an external device; sanitizing the distal end of an actuator component; recharging a power source of an actuator component; communicating with a computer, server, or database, and the like. The docking station may include a single actuator component dock, (i.e., a site or location configured to engagingly receive an actuator), or two or more actuator component docks, such that the number of actuator docks in a docking station may, in some instances, range from 1 to 6, such as 2 to 4. Docking stations of the invention may have any convenient configuration. Docking stations may be configured as table top devices, wall mounted devices, floor devices, etc., as desired. The component docks may be configured to only hold a cooling-power concentrator, which part can then be operably coupled to the device when another cooling power-concentrator is no longer at the desired temperature. The docking station may have several ports to charge several devices simultaneously, and may be configured to cool different devices that utilize the same mechanism of cooling or different mechanisms of cooling. In an embodiment, the base station may cool stand-alone cooling units that can be clipped into the device.

In order to provide different desired functionalities, the docking station may include a number of a different subsystems or components. For example, a docking station may include a cooling system, e.g., that is configured to maintain the temperature of docked actuator and/or therapeutic agent delivery components in a desired range. Examples of suitable cooling systems include, but are not limited, those described above. The docking station may include a communications module, e.g., for mediating data transfer between docked actuator and/or therapeutic agent delivery components and a module of the docking stations and/or an external device. The docking station may include a power module, e.g., for recharging a power source of a docked actuator. The docking station may include detector, e.g., for detecting docked actuator and/or therapeutic agent delivery components. The docking station may include an identifier reader, e.g., for reading an identifier on a docked actuator and/or therapeutic agent delivery components, such as an identifier reader as described above. Where desired, the docking station may include an identifier, such as described above.

In addition to docking stations, e.g., as described above, aspects of the invention further include docking systems. Docking systems include a docking station having one or more actuator components docked therewith.

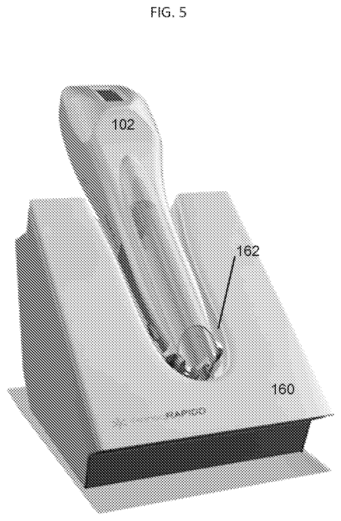

FIG. 5 provides a view docking station according to an embodiment of the invention. As shown in FIG. 5, docking station 160 includes a receiving space 162 for operably engaging an actuator component 102 of a device 100. Further details regarding docking station components are provided in PCT Application Serial No. PCT/US2018/037157; the disclosure of which is herein incorporated by reference.

### Smart Device Configurations

As described above, in some instances the active agent delivery system includes an identifier and the actuator component includes an identifier reader, e.g., as described above, such that the device may be viewed as a “smart” device. In such embodiments, a variety of different types of information may be stored on the identifier. Reading of the identifier by the identifier reader, e.g., when the therapeutic agent delivery component is release-ably engaged to the actuator component, transfers the information to the actuator component.

In some instances, the identifier includes active agent delivery component information. Active agent delivery component information is information or data about the active agent delivery component itself. Such information may include active agent delivery component historical information. Historical information is information about the nature of the active agent delivery component and/or one or more past events experienced by the active agent delivery component. Historical information includes, but is not limited to: an active agent identifier (e.g., the name of the therapeutic agent (or a proxy thereof) contained in the active agent delivery component), manufacturing lot number for active agent and/or active agent delivery component, active agent delivery component handling information (e.g., information about the supply channel through which the active agent delivery component has passed), active agent delivery component dose, concentration, and/or volume, and active agent delivery component expiration date, chain of custody information (e.g., shipment tracking information including time and geographical information, and temperature information over time, such as any storage temperature excursions that may have occurred, etc.) and the like. As such, historical information may include information about a particular active agent contained in an active agent delivery component. Such information may include, but is not limited to, identity of the active agent (brand name and/or generic name), date of manufacture, date of expiry, source of manufacture, dosage amount, drug concentration, intended route of administration, handling and storage information, delivery volume, indication for use, lot number, etc.

In some instances, the identifier includes active agent delivery component information that is active agent delivery component use information. Active agent delivery component use information is information or data about the actual use of the active agent delivery component, e.g., the actual employment of that active agent delivery component to deliver an active agent to a target delivery site. Such information may vary, and may include use date information (i.e., information about the data, time, etc., at which the component was used); administration information, e.g., confirmation that actual delivery to a subject occurred); identity of the subject to which the therapeutic agent was administer; condition of the subject for which the therapeutic agent was administered, etc.

As will be appreciated by the skilled artisan, this information could be stored directly on the identifier, or could be looked up in a linked database using the identifier information.

Smart device embodiments, e.g., as described above, allow for one or more desirable capabilities, including but not limited to inventory management capabilities, enhanced therapeutic capabilities, medical record history capabilities, data analytics capabilities, and the like. For example, a variety of different inventory management capabilities are provided by smart device embodiments, including automated reordering of therapeutic agent delivery component by a user (e.g., according to user preset preferences), tracking of individual therapeutic agent delivery components (e.g., to manage lost, stolen, or expired goods components), and the like. A variety of different enhanced therapeutic capabilities are provided by smart device embodiments, including auto-generation of procedure notes, communication with existing electronic medical records for integration in a patient chart, sending data on drug/dose/route for documentation purposes and/or billing purposes, aggregating data on drug/dose/route for market research and analytics, facilitating documentation for reimbursement, and the like. Examples of capabilities provided by smart device embodiments, e.g., as described herein, are further provided in Published United States Patent Application Publication Nos. 20160030683; 20170098058; 20170119969; 20170124284 and 20170124285; the disclosures of which capabilities are incorporated herein by references.

Examples of capabilities provided by smart device embodiments, e.g., as described herein, are further provided in PCT Application Serial No. PCT/US2018/037157; the disclosure of which is herein incorporated by reference.

### Methods

Aspects of the invention further include methods of delivering an active agent to a target tissue delivery site of a subject, e.g., by using an active agent delivery device of the invention. Aspects of the methods may include: contacting a sterile tissue contacting tip of an active agent delivery device, e.g., as described above, to the target tissue delivery site; and actuating the active agent delivery system to deliver a dosage of an active agent composition to the target tissue delivery site.

As reviewed above, the target tissue delivery site may vary. Examples of target tissue delivery sites include both external and internal delivery sites, wherein internal delivery sites include those sites located in body cavities. External sites include keratinized sites, as well as sites characterized by cutaneous membranes, mucous membranes, and tissue of the mucocutaneous zone. In some instances, the target tissue delivery site is an ocular site, where ocular sites of interest include a region that begins at the corneal limbus and extends anywhere from 1 mm to 10 mm posterior to the limbus, such as 2 mm to over 8 mm posterior to the limbus. In some instances, the area of interest includes the cornea and the corneal limbus.

To contact the distal end of the device with the target tissue site, the device may be manipulated so that the distal end of the device contacts the target tissue site. Where desired, contact of the distal end with the target tissue delivery site may be maintained by urging the distal end against the target tissue delivery site with moderate force.

As describe above, embodiments of devices may include a pain mitigation system. When present, the pain mitigation system may be actuated to mitigate pain at the target tissue delivery site. Depending on the nature of the pain mitigation system, the pain mitigation system may be activated before or after contact of the distal end of the device with the target tissue delivery site. For example, where the pain mitigation system is a cooling system, the pain mitigation system may be activated so that the sterile tissue contacting surface is at a desired temperature prior to contact of the sterile tissue contacting surface with the target tissue delivery site. Alternatively, where the pain mitigation system provides for pain mitigation via another mechanism, such as electrical stimulation, the pain mitigation system may be activated after contact of the sterile tissue contacting surface with the target tissue delivery site.

Following contact of the sterile tissue contacting surface with the target tissue delivery site, and actuation of a pain mitigation system if present, the active agent delivery system is actuated to deliver an amount, e.g., dosage, of an active agent to the target tissue delivery site. Where actuation of the active agent delivery system results in automated therapeutic agent delivery, the device is held in such a manner such that contact of the sterile tissue contacting surface with the target tissue delivery site is maintained during the active agent delivery. Following active agent delivery, the needle of the active agent delivery device may be withdrawn from the target tissue delivery site, e.g., using a controlled retraction profile.

The devices may be employed to deliver an active agent to a target tissue delivery site of a variety of different types of subjects. In some instances, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In certain embodiments, the subjects are humans. The methods may be diagnostic and/or therapeutic methods.

In some instances, the methods include assembling an active agent delivery device, e.g., by operably engaging an active agent delivery system in a receiving space of an actuator component to produce a complete device, e.g., as described above. In some instances, the method includes removing the actuator component from a docking station, such as described above. In some instances, the method further includes removing the active agent delivery system from the receiving space of the actuator component. The removing may include disposing the therapeutic agent delivery system. In some instances, the method further includes docking the actuator component in the docking station.

Aspects of the invention include methods of delivery an active agent into an eye of a subject. In such embodiments, the methods may include opening a packaging, e.g., as described below, which includes a syringe pre-filled with the active agent. As reviewed above, the syringe may have a sterile interior and the active agent present therein may be sterile. The syringe may also include a non-sterile exterior surface, e.g., at least a portion of the exterior surface of the body is non-sterile, e.g., as defined above. The methods may include operably coupling a sterile needle with the syringe, e.g., by coupling a sterile needle hub to the orifice of the syringe, e.g., via a luer fitting. In certain embodiments, the non-sterile exterior surface may then be contacted to one or more surfaces, such as but not limited to, a sterile field, sterile glove, an ocular surface of the patient, a surface of the needle or hub, etc., which renders the surface non-sterile, e.g., as defined above. Following this, the active agent may be administered into the eye, e.g., by depressing the plunger of the syringe.

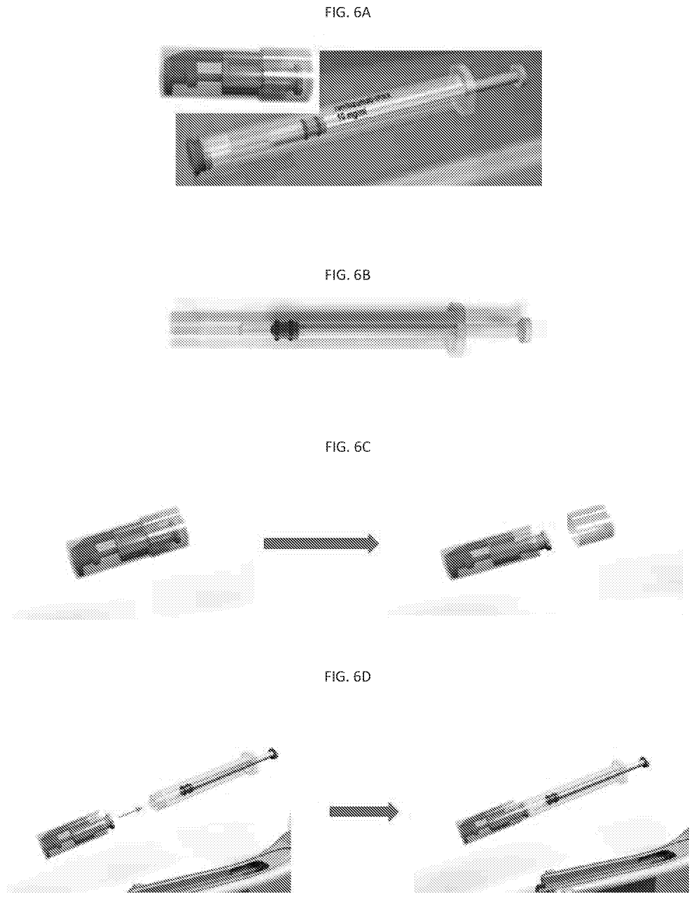

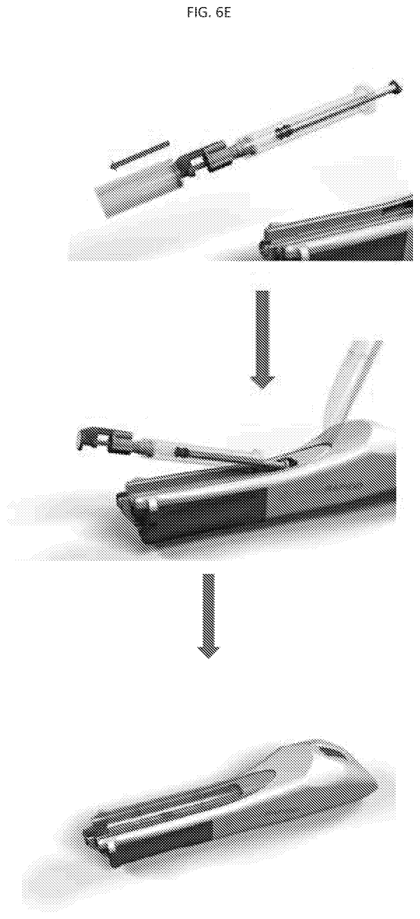

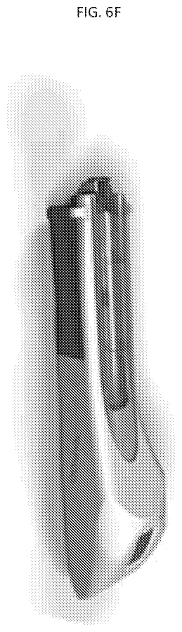

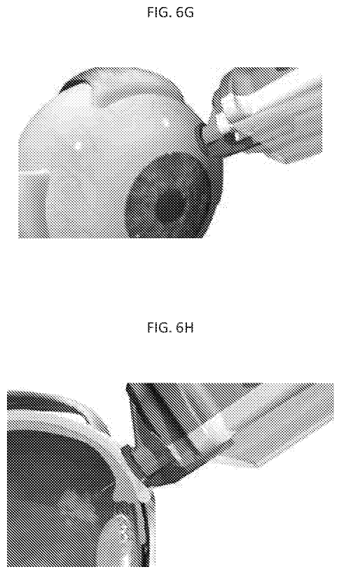

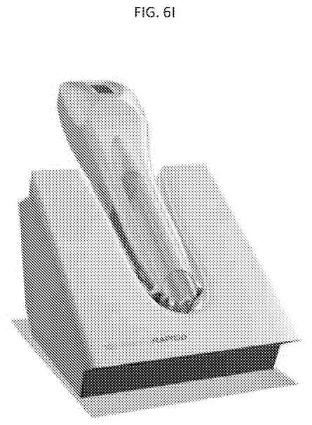

FIGS. 6A to 6I illustrate an active agent administration protocol according to an embodiment of the invention. In the protocol illustrated in FIGS. 6A to 6I, the protocol begins with provision of a tip present in a housing and a preloaded syringe having a removable cap or stopper at its distal end. The preloaded syringe is not surface sterilized and therefore may be handled outside of the sterile field. In the step illustrated in FIG. 6B, the distal end stopper is removed, making the syringe ready for operably engaging the tip. Removal of the stopper may expose a fitting, e.g., for operably coupling to the needle, where the fitting may be luer fitting, e.g., a luer slip or luer lock. In FIG. 6C, the cap of the tip housing is removed, exposing the proximal end of the tip. In FIG. 6D, the proximal end of the tip is coupled to the distal end of the syringe, e.g., via a press fit motion, such that the interior of the syringe is fluidically coupled to the needle of the tip and liquid active agent of the syringe may flow into the needle. In FIG. 6E, the tip container of the tip is removed, resulting in an active agent delivery system that is ready to be positioned into the receiving space of an actuator to produce an active agent delivery device 100. In FIG. 6F, the assembled device 100 is positioned in an upright position so as to prime the device, via an auto-priming activity of the actuator. Following priming of the device, the sterile tissue contacting surface of the device is contacted with the eye and maintained for a sufficient period of time for the cooling system to impart the desired pain mitigation, as shown in FIG. 6G. As shown in FIG. 6H, the actuator is activated to move the needle into the eye, e.g., where actuating includes moving the needle through a passageway of the sterile tissue contacting surface so that the distal end of the needle is positioned at the target tissue. Following placement of the distal end of the needle at the target tissue, the actuator then moves the plunger of the syringe so as to move the desired dosage of active agent composition from the syringe through the needle and into the eye. In some instances, the syringe is not present in the sterile field during actuation. Following use, the active agent delivery system is removed from the actuator and the actuator is placed in the docking station, e.g., as shown in FIG. 6I.

### Utility

Devices of the invention, e.g., as described above, find use in the delivery of a variety of different types of active agents to a target tissue delivery site to treat a variety of different types of conditions. The active agent delivery devices of the invention may be used to deliver an active agent to a variety of target tissue delivery sites. Examples of target tissue delivery sites include both external and internal delivery sites, wherein internal delivery sites include those sites located in body cavities. External sites may include keratinized sites, as well as sites characterized by cutaneous membranes, mucous membranes, and tissue of the mucocutaneous zone. In some instances, the target tissue delivery site is an ocular tissue delivery site, where ocular tissue delivery sites of interest include a region that begins at the corneal limbus and extends anywhere from 2 mm to over 8 mm posterior to the limbus, such as 3 mm to 6 mm from the corneal limbus, e.g., 3 to 4 mm from the corneal limbus, e.g., to allow intraocular injection via pars plana or pars plicata. Ocular tissue delivery sites may include conjunctiva, episclera, and sclera of the eye. In some instances, the subject devices are used for intravitreal injection therapy (IVT), retrobulbar injection therapy, subtenon injection therapy, subretinal injection therapy, suprachoroial injection, subconjunctival injection therapy, intracameral injection therapy, and the like.

The devices and methods of use may be employed to deliver a variety of different types of active agents. Any desired active agent composition may be delivered, where a given active agent composition may include a single active agent or combination of two or more difference active agents, as reviewed above. As reviewed above, an active agent is any component that provides pharmacological activity or other direct effect in the diagnosis, cure, mitigation, treatment, or prevention of disease, or affects the structure or any function of the body of man or animals. Active agents may vary, where examples of active agents include, but are not limited to, small molecule active agents, polypeptide active agents, e.g., antibodies and binding fragments thereof, fusion proteins, etc., nucleic acid active agents, cellular active agents, etc. Examples of therapeutic active agents that may be present include, but are not limited to: steroids, such as cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof; nonsteroidal anti-inflammatory agents such as salicylic-, indole acetic-, aryl acetic-, aryl propionic- and enolic acid derivatives including bromfenac, diclofenac, flurbiprofen, ketorolac tromethamine and nepafenac; antibiotic agents, such as bacitracin, besifloxacin, levofloxacin, moxifloxacin, sulfacetamide, tobramycin, cefazolin, cephradine, cefaclor, cephapirin, ceftizoxime, cefoperazone, cefotetan, cefuroxime, cefotaxime, cefadroxil, ceftazidime, cephalexin, cephalothin, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof; vascular endothelial growth factor (VEGF) modulators, e.g., VEGF inhibitors or antagonists, such as tyrosine kinase inhibitors, VEGF specific binding agents, e.g., VEGF antibodies or binding fragments thereof, VEGF binding fusion proteins, and the like; platelet derived growth factor (PDGF) modulators, e.g., PDGF inhibitors or antagonists, such as PDGF specific binding agents, e.g., PDGF antibodies or binding fragments thereof, PDGF binding fusion proteins, and the like; angiopoietin (ANG) modulators, such as ANG2 modulators, e.g., ANG2 inhibitors or antagonists, such as ANG2 specific binding agents, e.g., ANG2 antibodies or binding fragments thereof, ANG2 binding fusion proteins, and the like; combined ANG2 and VEGF inhibitors; antibody biopolymer conjugates containing VEGF inhibitors; poly-lactide-co-glycolide acid (PLGA) particles containing sunitinib malate; placental growth factor (PIGF) modulators, e.g., PIGF inhibitors or antagonists, such as PIGF specific binding agents, e.g., PIGF antibodies or binding fragments thereof, PIGF binding fusion proteins, and the like; tissue necrosis factor (TNF) modulators, such as anti-TNF alpha agents such as antibodies to TNF-α, antibody fragments to TNF-α and TNF binding fusion proteins including infliximab, etanercept, adalimumab, certolizumab and golimumab; mTOR inhibitors such as sirolimus, sirolimus analogues, Everolimus, Temsirolimus and mTOR kinase inhibitors; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents or intraocular pressure lowering agents, such as prostaglandin analogs, ROK inhibitors, beta blockers, alpha agonists, and carbonic anhydrase inhibitors; multi-specific modulators, e.g., bispecific modulators, such as bispecific binding agents, e.g., bispecific antibodies or binding fragments thereof, including agents that specifically bind to both VEGF and ANG2; aminosterols such as squalamine; antihistamines such as H 1-receptor antagonists and histamine H2-receptor antagonists, e.g., loratadine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimeorazine doxylamine, pheniramine, pyrilamine, chlorcyclizine, thonzylamine, and derivatives thereof; tyrosine kinase inhibitors, including receptor tyrosine kinase inhibitors; nucleic acid-based therapeutics such as gene vectors, e.g., plasmids, RNAi agents, e.g., siRNA, shRNA; complement system modulators, e.g., complement system inhibitors, including inhibitors of the alternative complement pathway, such as Factor D, properdin, Factor B, Factor Ba, and Factor Bb, and inhibitors of the classical complement pathway, such as C3a, C5, C5a, C5b, C6, C7, C8, C9 and C5b-9; chemotherapeutic agents, e.g., driamycin, cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof; insulin; etc.

The device may be employed to deliver a therapeutic agent to treat a variety of different disease conditions. Disease conditions of interest include, but are not limited to, ocular conditions, such as ocular disease conditions, such as intraocular neovascular disease conditions. An “intraocular neovascular disease” is a disease characterized by ocular neovascularisation. Examples of intraocular neovascular diseases include, for example, proliferative retinopathies, choroidal neovascularization (CNV), age-related macular degeneration (AMD), geographic atrophy (GA), diabetic and other ischemia-related retinopathies, diabetic macular edema, pathological myopia, von Hippel-Lindau disease, histoplasmosis of the eye, Central Retinal Vein Occlusion (CRVO), Branch Retinal Vein Occlusion (BRVO), pterygium, corneal neovascularization, and retinal neovascularization. The term “age-related macular degeneration” refers to a medical condition which usually affects older adults and results in a loss of vision in the center of the visual field (the macula) because of damage to the retina. Some or all of these conditions can be treated by intravitreal injection of a VEGF-antagonist, e.g., as described above. Other ocular conditions that may be treated in accordance with aspects of the invention include, but are not limited to: retinal detachments (pneumatic retinopexy), by using devices of the invention to inject a gas into the eye, where the device may control the depth of injection to a desired/optimal depth. Disease conditions of interest also include central serous chorioretinopathy and uveitis, including anterior uveitis, pars planitis, intermediate uveitis, and posterior uveitis.

### Kits

Also provided are kits that include at least one or more therapeutic agent delivery components, e.g., as described above. For example, a kit may include a needle and a tissue contacting tip, e.g., as described above and/or a syringe prefilled with an active agent composition, where these components may be separate or operably engaged with each other as a composite structure. For example, a kit may include a tip component that includes a tip engaged with a needle and present in a housing (e.g., as illustrated in FIG. 3B) and a prefilled syringe as illustrated in FIG. 4B. A kit may further include, where desired, an actuator component, a docking station, etc. The kit components may be present in packaging, which packaging may or may not be sterile, as desired. For example, where the kit includes a prefilled syringe, e.g., as described above, the packaging may not be exposed to sterilization following placement of the syringe in the packaging.

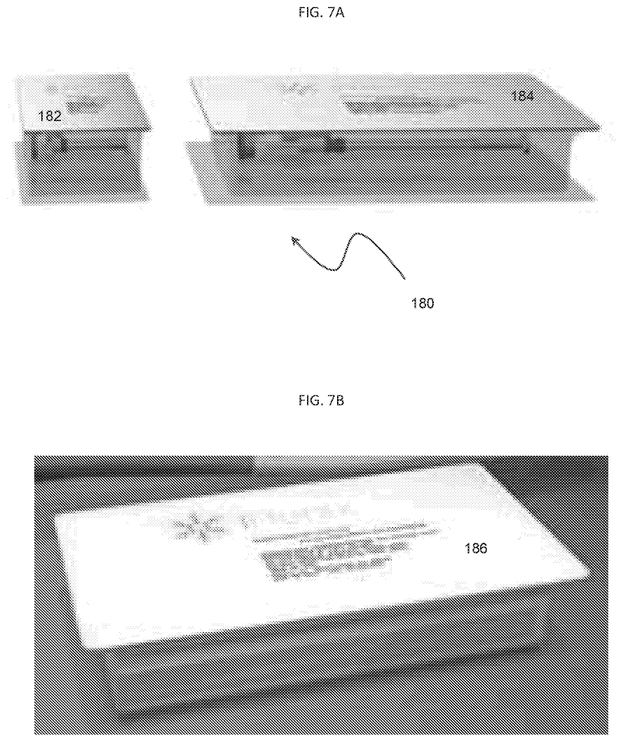

FIGS. 7A and 7B provide views of kits according to two different embodiments. In FIG. 7A, the kit 180 includes a first packaged component 182 having present therein a tip component, such as shown in FIG. 3B, and a second packaged component 184 having present therein a prefilled syringe, such as shown in FIG. 4B. In FIG. 7B, the kit includes a single packaged component 186 having both the tip component and the prefilled syringe present therein.

Also present in the kit may be instructions for using the kit components. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g., portable flash drive, DVD- or CD-ROM, etc. The instructions may take any form, including complete instructions for how to use the device or as a website address with which instructions posted on the world wide web may be accessed.

Notwithstanding the appended claims, the disclosure is also defined by the following clauses:

1. An active agent delivery device, the device comprising:

(a) a syringe that is not surface sterilized and comprises a liquid composition of the active agent;

(b) a tip comprising a sterile tissue contacting surface; and

(c) a needle operably coupled to the syringe and the tip.

2. The device according to Clause 1, wherein the syringe has a volume ranging from 0.1 to 5.0 ml.
3. The device according to Clause 2, wherein the syringe has a volume ranging from 0.1 to 1.50 ml.
4. The device according to Clause 3, wherein the syringe has a volume ranging from 0.1 to 1.0 ml.
5. The device according to any of the preceding clauses, wherein the device is configured to deliver a dosage having a volume ranging from 5 μl to 100 μl.
6. The device according to Clause 5, wherein the device is configured to deliver a dosage having a volume ranging from 10 μl to 50 μl.
7. The device according to any of the preceding clauses, wherein the syringe comprises a dosing mark.
8. The device according to Clause 7, wherein the dosing mark indicates delivery of a dosage that is less than the volume of the syringe.
9. The device according to Clauses 7 or 8, wherein the dosing mark ranges from 5 to 100 μL.
10. The device according to Clause 9, wherein the dosing mark is 50 μL.
11. The device according to any of Clauses 1 to 6, wherein the syringe does not include a dosing mark.
12. The device according to any of the preceding clauses, wherein the syringe comprises one or more chambers.
13. The device according to any of the preceding clauses, wherein the syringe comprises a glass.
14. The device according to any of Clauses 1 to 12, wherein the syringe comprises a plastic.
15. The device according to Clause 14, wherein the plastic is selected from the group consisting of cyclic olefin polymer (COP) and cyclic olefin copolymer (COC).
16. The device according to any of the preceding clauses, wherein the sterile tissue contacting surface is planar.
17. The device according to any of the preceding clauses, wherein the sterile tissue contacting surface has a tissue contacting surface area ranging from 0.5 to 50 mm².
18. The device according to any of the preceding clauses, wherein the sterile tissue contacting surface comprises a passageway dimensioned to accommodate passage of the needle therethrough.
19. The delivery device according to any of the preceding clauses, wherein the needle has a gauge ranging from 23 to 35.
20. The delivery device according to Clause 19, wherein the needle has a gauge ranging from 30 to 33.
21. The delivery device according to Clause 19, wherein the needle has a gauge ranging from 23 to 30.
22. The device according to any of Clauses 19 to 21, wherein the needle comprises two or more lumens.
23. The device according to Clause 22, wherein the needle comprises two lumens.
24. The device according to any of the preceding clauses, wherein the distal end of the needle is movable relative to the sterile tissue contacting surface.
25. The device according to any of the preceding clauses, wherein the needle is present in a needle housing.
26. The device according to Clause 25, wherein the tip is operably coupled to the distal end of the needle housing and the distal end of the syringe is operably coupled to the proximal end of the needle housing.
27. The device according to Clause 26, wherein the distal end of the syringe is operably coupled to the proximal end of the needle housing by a fitting.
28. The device according to Clause 27, wherein the fitting is a luer fitting.
29. The device according to Clause 28, wherein the luer fitting comprises a luer slip.
30. The device according to Clause 28, wherein the luer fitting comprises a luer lock.
31. The device according to any of the preceding clauses, wherein the tip is operably coupled to two or more needles.
32. The device according to any of the preceding clauses, wherein the device further comprises an actuator component comprising an actuator configured to move the liquid composition through the needle.
33. The device according to Clause 32, wherein the actuator comprises a motor.
34. The device according to Clause 32, wherein the actuator comprises a spring.
35. The device according to Clause 32, wherein the actuator comprises a manual actuator.
36. The device according to any of Clauses 32 to 35, wherein the actuator is configured to sequentially perform a first priming motion and a second injection motion.
37. The device according to Clause 36, wherein the actuator is further configured to withdraw the needle following the second injection motion.
38. The device according to any of Clauses 32 to 37, wherein the actuator component further comprises a syringe receiving space comprising the syringe.
39. The device according to any of Clauses 32 to 38, wherein the actuator component further comprises a pain mitigation system operatively coupled to the tip.
40. The device according to Clause 39, wherein the pain mitigation system comprises an anesthesia producing system.
41. The device according to Clause 40, wherein the anesthesia producing system comprises a cooling system.
42. The device according to Clause 41, wherein the cooling system is selected from the group consisting of: a thermoelectric cooling system, a liquid evaporation cooling system, a solid sublimation cooling system, a solid melting cooling system, a Joule-Thompson cooling system, a thermodynamic cycle cooling system, an endothermic reaction cooling system and a low-temperature substance cooling system.
43. The device according to Clause 42, wherein the cooling system comprises a thermoelectric cooling system.
44. The device according to Clause 43, wherein the thermoelectric cooling system comprises a Peltier unit, a conductor coupling the Peltier unit to the tissue contacting surface and a heat sink coupled to the Peltier unit.
45. The device according to Clause 42, wherein the cooling system comprises a low-temperature substance cooling system.
46. The device according to Clause 45, wherein the low-temperature substance comprises a liquid or gel.
47. The device according to any of the preceding clauses, wherein the liquid composition comprises a VEGF modulator.
48. The device according to Clause 47, wherein the VEGF modulator comprises an antibody, bispecific antibody, or binding fragment thereof.
49. The device according to any of the preceding clauses, wherein the liquid composition comprises a complement system modulator.
50. The device according to Clause 49, wherein the complement system modulator comprises a small molecule, antibody or binding fragment thereof.
51. The device according to any of the preceding clauses, wherein the liquid composition comprises an intraocular pressure lowering agent.
52. The device according to Clause 51, wherein the intraocular pressure lowering agent is selected from the group consisting prostaglandin analogs, ROK inhibitors, beta blockers, carbonic anhydrase inhibitors, and alpha agonists, tyrosine kinase inhibitors, angiopoietin inhibitors, placental growth factor inhibitors, nucleic acid agent, and combinations thereof.
53. The device according to any of the preceding clauses, wherein the liquid composition comprises two or more active agents.
54. The device according to Clause 53, wherein the liquid composition comprises a VEGF modulator and a complement system modulator.
55. The device according to Clause 54, wherein the liquid composition comprises a bispecific antibody and a complement system modulator.
56. The device according to any of the preceding clauses, wherein the syringe comprises two or more chambers each containing a distinct liquid composition.
57. The device according to Clause 56, wherein the distinct liquid compositions have different viscosities.
58. The device according to any of Clauses 1 to 55, wherein the device comprises two or more syringes each containing a distinct liquid composition.
59. The device according to Clause 58, wherein the distinct liquid compositions have different viscosities.
60. The device according to any of the preceding clauses, wherein the device is a handheld device.
61. The device according to any of the preceding clauses, wherein the device is configured to deliver the liquid composition to an ocular tissue delivery site.
62. A tip component for an active agent delivery device, the tip component comprising:

a tip comprising a sterile tissue contacting surface; and

a needle operably coupled to the tip.

63. The tip component according to Clause 62, wherein the sterile tissue contacting surface is planar.
64. The tip component according to any of Clauses 62 and 63, wherein the sterile tissue contacting surface has a tissue contacting surface area ranging from 0.5 to 50 mm².
65. The tip component according to any of Clauses 62 to 64, wherein the sterile tissue contacting surface comprises a passageway dimensioned to accommodate passage of the needle therethrough.
66. The tip component according to any of Clauses 62 to 65, wherein the needle has a gauge ranging from 23 to 35.
67. The tip component according to Clause 66, wherein the needle has a gauge ranging from 30 to 33.
68. The tip component according to Clause 66, wherein the needle has a gauge ranging from 23 to 30.
69. The tip component according to any of Clauses 66 to 68, wherein the needle comprises two or more lumens.
70. The tip component according to Clause 69, wherein the needle comprises two lumens.
71. The tip component according to any of Clauses 62 to 70, wherein the distal end of the needle is movable relative to the sterile tissue contacting surface.
72. The tip component according to any of Clauses 62 to 71, wherein the needle is present in a needle housing.
73. The tip component according to Clause 72, wherein the tip is operably coupled to the distal end of the needle housing.
74. The tip component according to Clause 73, wherein the proximal end of the needle housing comprises a fitting.
75. The tip component according to Clause 74, wherein the fitting is a luer fitting.
76. The tip component according to Clause 75, wherein the luer fitting comprises a luer slip.
77. The tip component according to Clause 75, wherein the luer fitting comprises a luer lock.
78. The tip component according to any of Clauses 62 to 77, wherein the tip component is present in a sealed housing.
79. The tip component according to Clause 78, wherein the sealed housing comprises a sterile interior and a non-sterile exterior.
80. The tip component according to any of Clauses 78 and 79, wherein the housing comprises a tip component container and cap.
81. The tip component according to any of Clauses 62 to 80, wherein the tip is operably coupled to two or more needles.
82. A syringe that is not surface sterilized and comprises a sterile liquid composition comprising an active agent.
83. The syringe according to Clause 82, wherein the syringe has a volume ranging from 0.1 to 5.0 ml.
84. The syringe according to Clause 83, wherein the syringe has a volume ranging from 0.1 to 1.50 ml.
85. The syringe according to Clause 84, wherein the syringe has a volume ranging from 0.1 to 1.0 ml.
86. The syringe according to any of Clauses 82 to 85, wherein the syringe is configured to deliver a dosage having a volume ranging from 5 μl to 100 μl.
87. The syringe according to Clause 86, wherein the syringe is configured to deliver a dosage having a volume ranging from 10 μl to 50 μl.
88. The syringe according to any of Clauses 82 to 87, wherein the syringe comprises a dosing mark.
89. The syringe according to Clause 88, wherein the dosing mark indicates delivery of a dosage that is less than the volume of the syringe.
90. The syringe according to Clauses 88 or 89, wherein the dosing mark ranges from 10 to 100 μL.
91. The syringe according to Clause 90, wherein the dosing mark is 50 μL.
92. The syringe according to any of Clauses 82 to 87, wherein the syringe does not include a dosing mark.
93. The syringe according to any of Clauses 82 to 92, wherein the liquid composition comprises a VEGF modulator.
94. The syringe according to Clause 93, wherein the VEGF modulator comprises an antibody, bispecific antibody or binding fragment thereof.
95. The syringe according to any of Clauses 82 to 94, wherein the liquid composition comprises a complement system modulator.
96. The syringe according to Clause 95, wherein the complement system modulator comprises a small molecule, antibody or binding fragment thereof.
97. The syringe according to any of Clauses 82 to 96, wherein the liquid composition comprises an intraocular pressure lowering agent.
98. The syringe according to Clause 97, wherein the intraocular pressure lowering agent is selected from the group consisting prostaglandin analogs, ROK inhibitors, beta blockers, carbonic anhydrase inhibitors, and alpha agonists, tyrosine kinase inhibitors, angiopoietin inhibitors, placental growth factor inhibitors, nucleic acid agent, and combinations thereof.
99. The syringe according to any of Clauses 82 to 98, wherein the liquid composition comprises two or more active agents.
100. The syringe according to Clause 99, wherein the liquid composition comprises a VEGF modulator and a complement system modulator.
101. The syringe according to Clause 100, wherein the liquid composition comprises a bispecific antibody and a complement system modulator.
102. The syringe according to any of Clauses 82 to 101, wherein the syringe comprises two or more chambers each containing a distinct liquid composition.
103. The syringe according to Clause 102, wherein the distinct liquid compositions have different viscosities.
104. The syringe according to any of Clauses 82 to 103, wherein the syringe comprises a distal end removable cap.
105. The syringe according to Clause 104, wherein removal of the removable cap exposes a fitting.
106. The syringe according to Clause 105, wherein the fitting comprises a luer fitting.
107. The syringe according to Clause 106, wherein the luer fitting comprises a luer slip.
108. The syringe according to Clause 106, wherein the luer fitting comprises a luer lock.
109. The syringe according to any of Clauses 82 to 108, wherein the syringe comprises one or more chambers.
110. The syringe according to any of Clauses 82 to 109, wherein the syringe comprises a glass.
111. The syringe according to any of Clauses 82 to 109, wherein the syringe comprises a plastic.
112. The syringe according to Clause 111, wherein the plastic is selected from the group consisting of cyclic olefin polymer (COP) and cyclic olefin copolymer (COC).
113. An actuator component for an active agent delivery device comprising a syringe that is not surface sterilized and comprises a liquid composition of the active agent, a tip operably comprising a sterile tissue contacting surface, and a needle operably coupled to the syringe and tip, the actuator component comprising:

an actuator configured to move the liquid composition through the needle.

114. The actuator component according to Clause 113, wherein the actuator comprises a motor.
115. The actuator component according to Clause 113, wherein the actuator comprises a spring.
116. The actuator component according to Clause 113, wherein the actuator comprises a manual actuator.
117. The actuator component according to any of Clauses 113 to 116, wherein the actuator is configured to sequentially perform a first priming motion and a second injection motion.
118. The actuator component according to Clause 117, wherein the actuator is further configured to withdraw the needle following the second injection motion.
119. The actuator component according to any of Clauses 113 to 118, wherein the actuator component further comprises a syringe receiving space configured to receive the syringe.
120. The actuator component according to any of Clauses 113 to 119, wherein the actuator component further comprises a pain mitigation system operatively coupled to the tip.
121. The actuator component according to Clause 120, wherein the pain mitigation system comprises an anesthesia producing system.
122. The actuator component according to Clause 121, wherein the anesthesia producing system comprises a cooling system.
123. The actuator according to Clause 122, wherein the cooling system is selected from the group consisting of: a thermoelectric cooling system, a liquid evaporation cooling system, a solid sublimation cooling system, a solid melting cooling system, a Joule-Thompson cooling system, a thermodynamic cycle cooling system, an endothermic reaction cooling system and a low-temperature substance cooling system.
124. The actuator according to Clause 123, wherein the cooling system comprises a thermoelectric cooling system.
125. The actuator according to Clause 124, wherein the thermoelectric cooling system comprises a Peltier unit, a conductor coupling the Peltier unit to the tissue contacting surface and a heat sink coupled to the Peltier unit.
126. The actuator according to Clause 123, wherein the cooling system comprises a low-temperature substance cooling system.
127. The actuator according to Clause 126, wherein the low-temperature substance comprises a liquid or gel.
128. The actuator component according to any of Clauses 113 to 127, wherein the actuator component is present in a docking station.
129. A method of delivering one or more active agents to a target tissue, the method comprising:

(a) contacting a sterile tissue contacting surface of an active agent delivery device comprising:
(i) a syringe that is not surface sterilized and comprises a liquid composition of the one or more active agents;
(ii) a tip comprising the sterile tissue contacting surface; and
(iii) a needle operably coupled to the syringe and tip;

with a target tissue delivery site for the target tissue; and

(b) actuating the syringe to move a dose of the liquid composition from the syringe through the needle so as to deliver the active agent to the target tissue.

130. The method according to Clause 129, wherein actuating comprises moving the needle through a passageway of the sterile tissue contacting surface so that the distal end of the needle is positioned at the target tissue.
131. The method according to any of Clauses 129 to 130, wherein the method further comprises priming the device.
132. The method according to any of Clauses 129 to 131, wherein method further comprising assembling the device.
133. The method according to Clause 132, wherein assembling the device comprises operably coupling the syringe to a tip component comprising the tip and the needle to produce an assembled syringe and needle.
134. The method according to any of Clauses 129 to 133, wherein the actuation comprises manual actuation.
135. The method according to Clause 134, wherein the manual actuation comprises sequentially moving the needle relative to the tip and then depressing a plunger of the syringe.
136. The method according to Clause 135, wherein the method further comprises operably coupling the assembled syringe and needle to an actuator.
137. The method according to Clause 136, wherein actuator further comprises a pain mitigation system and the method further comprises actuating a pain mitigation system.
138. The method according to any of Clauses 129 to 137, wherein the liquid composition comprises a VEGF modulator.
139. The method according to Clause 138, wherein the VEGF modulator comprises an antibody, bispecific antibody or binding fragment thereof.
140. The method according to any of Clauses 129 to 139, wherein the liquid composition comprises a complement system modulator.
141. The method according to Clause 140, wherein the complement system modulator comprises a small molecule, antibody or binding fragment thereof.
142. The method according to any of Clauses 129 to 141, wherein the liquid composition comprises an intraocular pressure lowering agent.
143. The method according to Clause 142, wherein the intraocular pressure lowering agent is selected from the group consisting prostaglandin analogs, ROK inhibitors, beta blockers, carbonic anhydrase inhibitors, and alpha agonists, tyrosine kinase inhibitors, angiopoietin inhibitors, placental growth factor inhibitors, nucleic acid agent, and combinations thereof.
144. The method according to any of Clauses 129 to 143, wherein the liquid composition comprises two or more active agents.
145. The method according to Clause 144, wherein the liquid composition comprises a VEGF modulator and a complement system modulator.
146. The method according to Clause 144, wherein the liquid composition comprises a bispecific antibody and a complement system modulator.
147. The method according to any of Clauses 129 to 146, wherein the syringe comprises two or more chambers each containing a distinct liquid composition.
148. The method according to Clause 147, wherein the distinct liquid compositions have different viscosities.
149. The method according to any of Clauses 129 to 148, wherein the syringe is not present in the sterile field during contacting and actuating.
150. The method according to Clause 149, wherein the method comprises handling the syringe in a non-sterile manner.
151. The method according to any of Clauses 129 to 150, wherein the target tissue delivery site comprises an ocular tissue delivery site.
152. The method according to Clause 151, wherein the ocular tissue delivery site is positioned within 0.5 mm to 4 mm of the limbus.
153. The method according to Clause 152, wherein the method comprises delivery the liquid composition to the anterior chamber via the cornea.
154. The method according to any of Clauses 129 to 153, wherein the method is a method of treating a subject for an ocular disease.
155. A method of delivering an active agent composition into an eye of a subject, the method comprising:

(a) opening a packaging containing a pre-filled syringe comprising:
(i) a sterile interior comprising the active agent composition; and
(ii) a non-sterile exterior surface;

(b) operably coupling a sterile hub of a sterile needle to the syringe;

(c) contacting the non-sterile exterior surface to a second surface such that the second surface is non-sterile following the contacting; and

(d) administering the active agent composition into the eye of the subject with the syringe.

156. The method according to Clause 155, wherein the method is a method of treating a subject for an ocular disease.
157. The method according to Clause 156, wherein the active agent composition comprises a VEGF modulator.
158. The method according to Clause 156, wherein the VEGF modulator comprises an antibody, bispecific antibody or binding fragment thereof.
159. The method according to Clause 156, wherein the active agent comprises a complement system modulator.
160. The method according to Clause 159, wherein the complement system modulator comprises a small molecule, antibody or binding fragment thereof.
161. The method according to Clause 156, wherein the active agent comprises an intraocular pressure lowering agent.
162. The method according to Clause 161, wherein the intraocular pressure lowering agent is selected from the group consisting prostaglandin analogs, ROK inhibitors, beta blockers, carbonic anhydrase inhibitors, and alpha agonists, tyrosine kinase inhibitors, angiopoietin inhibitors, placental growth factor inhibitors, nucleic acid agent, and combinations thereof.
163. The method according to any of Clauses 155 to 162, wherein the active agent composition comprises two or more active agents.
164. The method according to Clause 163, wherein the active agent composition comprises a VEGF modulator and a complement system modulator.
165. The method according to Clause 163, wherein the active agent composition comprises a bispecific antibody and a complement system modulator.
166. The method according to any of Clauses 155 to 165, wherein the syringe comprises two or more chambers each containing a distinct active agent composition.
167. The method according to Clause 166, wherein the distinct liquid compositions have different viscosities.
168. The method according to any of Clauses 155 to 167, wherein the needle has a gauge ranging from 23 to 35.
169. The method device according to Clause 168, wherein the needle has a gauge ranging from 29 to 33.
170. The method according to any of Clauses 155 to 169, wherein the second surface comprises a sterile field.
171. The method according to any of Clauses 155 to 170, wherein the second surface comprises a sterile glove.
172. The method according to any of Clauses 155 to 171, wherein the second surface comprises an ocular surface.
173. The method according to any of Clauses 155 to 172, wherein the second surface comprises a surface of the needle or needle hub.
174. A kit comprising:

(a) an active agent delivery device tip component comprising:
(i) a tip comprising a sterile tissue contacting surface; and
(ii) a needle operably coupled to the tip;

wherein the tip component is present in a sealed housing comprising a sterile interior and non-sterile exterior; and

(b) a syringe that is not surface sterilized and comprises a liquid composition comprising an active agent.

175. The kit according to Clause 174, wherein the needle is sterile.
176. The kit according to any of Clauses 174 to 175, wherein the sterile tissue contacting surface is planar.
177. The kit according to any of Clauses 174 to 176, wherein the sterile tissue contacting surface has a tissue contacting surface area ranging from 0.5 to 50 mm².
178. The kit according to any of Clauses 174 to 177, wherein the sterile tissue contacting surface comprises a passageway dimensioned to accommodate passage of the needle therethrough.
179. The kit according to any of Clauses 174 to 178, wherein the needle has a gauge ranging from 23 to 35.
180. The kit according to Clause 179, wherein the needle has a gauge ranging from 30 to 33.
181. The kit according to Clause 180, wherein the needle has a gauge ranging from 23 to 30.
182. The kit according to any of Clauses 174 to 181, wherein the needle comprises two or more lumens.
183. The kit according to Clause 182, wherein the needle comprises two lumens.
184. The kit according to any of Clauses 174 to 183, wherein the distal end of the needle is movable relative to the sterile tissue contacting surface.
185. The kit according to any of Clauses 174 to 184, wherein the needle is present in a needle housing.
186. The kit according to Clause 185, wherein the tip is operably coupled to the distal end of the needle housing.
187. The kit according to Clause 186, wherein the proximal end of the needle housing comprises a fitting.
188. The kit according to Clause 187, wherein the fitting is a luer fitting.
189. The kit according to Clause 188, wherein the luer fitting comprises a luer slip.
190. The kit according to Clause 188, wherein the luer fitting comprises a luer lock.
191. The kit according to any of Clauses 184 to 190, wherein the housing comprises a tip component container and cap.
192. The kit according to any of Clauses 174 to 191, wherein the syringe has a volume ranging from 0.1 to 5.0 ml. 193. The kit according to Clause 192, wherein the syringe has a volume ranging from 0.1 to 1.50 ml.
194. The kit according to Clause 193, wherein the syringe has a volume ranging from 0.1 to 1.0 ml.
195. The kit according to any of Clauses 174 to 194, wherein the syringe is configured to deliver a dosage having a volume ranging from 5 μl to 100 μl.
196. The kit according to Clause 195, wherein the syringe is configured to deliver a dosage having a volume ranging from 10 μl to 50 μl.
197. The kit according to any of Clauses 174 to 196, wherein the syringe comprises a dosing mark.
198. The kit according to Clause 197, wherein the dosing mark indicates delivery of a dosage that is less than the volume of the syringe.
199. The kit according to any of Clauses 197 to 198, wherein the dosing mark ranges from 10 to 100 μL.
200. The kit according to Clause 199, wherein the dosing mark is 50 μL.
201. The kit according to any of claims 174 to 196, wherein the kit does not include a dosing mark.
202. The kit according to any of Clauses 174 to 201, wherein the liquid composition comprises a VEGF modulator.
203. The kit according to Clause 202, wherein the VEGF modulator comprises an antibody, bispecific antibody or binding fragment thereof.
204. The kit according to any of Clauses 174 to 203, wherein the liquid composition comprises a complement system modulator.
205. The kit according to Clause 204, wherein the complement system modulator comprises a small molecule, antibody or binding fragment thereof.
206. The kit according to any of Clauses 174 to 205, wherein the liquid composition comprises an intraocular pressure lowering agent.
207. The kit according to Clause 206, wherein the intraocular pressure lowering agent is selected from the group consisting prostaglandin analogs, ROK inhibitors, beta blockers, carbonic anhydrase inhibitors, and alpha agonists, tyrosine kinase inhibitors, angiopoietin inhibitors, placental growth factor inhibitors, nucleic acid agent, and combinations thereof.
208. The kit according to any of Clauses 174 to 207, wherein the liquid composition comprises two or more active agents.
209. The kit according to Clause 208, wherein the liquid composition comprises a VEGF modulator and a complement system modulator.
210. The kit according to Clause 208, wherein the liquid composition comprises a bispecific antibody and a complement system modulator.
211. The kit according to any of Clauses 174 to 210, wherein the syringe comprises two or more chambers each containing a distinct liquid composition.
212. The kit according to Clause 211, wherein the distinct liquid compositions have different viscosities.
213. The kit according to any of Clauses 174 to 212, wherein the syringe comprises a distal end removable cap.
214. The kit according to Clause 213, wherein removal of the removable cap exposes a fitting.
215. The kit according to Clause 214, wherein the fitting comprises a luer fitting.
216. The kit according to Clause 215, wherein the luer fitting comprises a luer slip.
217. The kit according to Clause 215, wherein the luer fitting comprises a luer lock.
218. A kit comprising:

(a) a syringe comprising:
(i) a body having a non-sterile exterior surface and defining a sterile interior space comprising an active agent composition;
(ii) a plunger at a first end of the body;
(iii) an orifice at a second end of the body; and
(iv) a removable cap sealing the orifice; and

(b) non-sterile packaging containing the syringe.

219. The kit according to Clause 218, wherein the non-sterile packaging has not been exposed to sterilization following placement of the syringe in the packaging.
220. The kit according to any of Clauses 218 to 219, wherein the syringe has a volume ranging from 0.1 to 5.0 ml.
221. The kit according to Clause 220, wherein the syringe has a volume ranging from 0.1 to 1.50 ml.
222. The kit according to Clause 221, wherein the syringe has a volume ranging from 0.1 to 1.0 ml.
223. The kit according to any of Clauses 218 to 222, wherein the syringe is configured to deliver a dosage having a volume ranging from 5 μl to 100 μl.
224. The kit according to Clause 223, wherein the syringe is configured to deliver a dosage having a volume ranging from 10 μl to 50 μl.
225. The kit according to any of Clauses 218 to 224, wherein the syringe comprises a dosing mark.
226. The kit according to Clause 225, wherein the dosing mark indicates delivery of a dosage that is less than the volume of the syringe.
227. The kit according to Clauses 225 to 226, wherein the dosing mark ranges from 10 to 100 μL.
228. The kit according to Clause 227, wherein the dosing mark is 50 μL.
229. The kit according to any of Clauses 218 to 224, wherein the syringe does not include a dosing mark.
230. The kit according to any of Clauses 218 to 229, wherein the active agent composition comprises a VEGF modulator.
231. The kit according to Clause 230, wherein the VEGF modulator comprises an antibody, bispecific antibody or binding fragment thereof.
232. The kit according to any of Clauses 218 to 229, wherein the active agent composition comprises a complement system modulator.
233. The kit according to Clause 232, wherein the complement system modulator comprises a small molecule, antibody or binding fragment thereof.
234. The kit according to any of Clauses 218 to 229, wherein the active agent composition comprises an intraocular pressure lowering agent.
235. The kit according to Clause 234, wherein the intraocular pressure lowering agent is selected from the group consisting prostaglandin analogs, ROK inhibitors, beta blockers, carbonic anhydrase inhibitors, and alpha agonists, tyrosine kinase inhibitors, angiopoietin inhibitors, placental growth factor inhibitors, nucleic acid agent, and combinations thereof.
236. The kit according to any of Clauses 218 to 235, wherein the liquid composition comprises two or more active agents.
237. The kit according to Clause 236, wherein the liquid composition comprises a VEGF modulator and a complement system modulator.
238. The kit according to Clause 236, wherein the liquid composition comprises a bispecific antibody and a complement system modulator.
239. The kit according to any of Clauses 218 to 238, wherein the syringe comprises two or more chambers each containing a distinct liquid composition.
240. The kit according to Clause 239, wherein the distinct liquid compositions have different viscosities.
241. The kit according to any of Clauses 218 to 240, wherein removal of the removable cap exposes a fitting.
242. The kit according to Clause 241, wherein the fitting comprises a luer fitting.
243. The kit according to Clause 242, wherein the luer fitting comprises a luer slip or a luer lock.
244. The kit according to any of Clauses 218 to 243, where the syringe body is made of glass.
245. The kit according to any of Clauses 218 to 243, where the syringe is made of plastic

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. An active agent delivery device, the device comprising:
(a) a syringe that is not surface sterilized and comprises a liquid composition of the active agent;
(b) a tip comprising a sterile tissue contacting surface; and
(c) a needle operably coupled to the syringe and the tip.

2. The device according to claim 1, wherein the syringe has a volume ranging from 0.1 to 5.0 ml.

3. The device according to any of the preceding claims, wherein the device is configured to deliver a dosage having a volume ranging from 5 μl to 100 μl.

4. The device according to any of the preceding claims, wherein the syringe comprises a dosing mark.

5. The device according to any of the preceding claims, wherein the sterile tissue contacting surface comprises a passageway dimensioned to accommodate passage of the needle therethrough.

6. The device according to any of the preceding claims, wherein the distal end of the needle is movable relative to the sterile tissue contacting surface.

7. The device according to any of the preceding claims, wherein the needle is present in a needle housing.

8. The device according to claim 7, wherein the tip is operably coupled to the distal end of the needle housing and the distal end of the syringe is operably coupled to the proximal end of the needle housing.

9. The device according to any of the preceding claims, wherein the device further comprises an actuator component comprising an actuator configured to move the liquid composition through the needle.

10. The device according to claim 9, wherein the actuator component further comprises a pain mitigation system operatively coupled to the tip.

11. A tip component for an active agent delivery device according to any of claims 1 to 10.

12. A syringe that is not surface sterilized and comprises a sterile liquid composition comprising an active agent as described in any of claims 1 to 10.

13. An actuator component for an active agent delivery device as described in any of claims 1 to 10.

14. A method of delivering one or more active agents to a target tissue, the method comprising:
(a) contacting a sterile tissue contacting surface of an active agent delivery device according to any of claims 1 to 10;
with a target tissue delivery site for the target tissue; and
(b) actuating the syringe to move a dose of the liquid composition from the syringe through the needle so as to deliver the active agent to the target tissue.

15. A kit comprising:
(a) an active agent delivery device tip component comprising:
(i) a tip comprising a sterile tissue contacting surface; and
(ii) a needle operably coupled to the tip;
wherein the tip component is present in a sealed housing comprising a sterile interior and non-sterile exterior; and
(b) a syringe that is not surface sterilized and comprises a liquid composition comprising an active agent.
